(19) United States
(12) Patent Application Publication
(10) Pub. No.: US 2011/0251848 A1
(43) Pub. Date: Oct. 13, 2011

(54) GLOBAL HEALTH CARE COMMUNITY AND MEDICAL RECORD ACCESS WEBSITE

(75) Inventors: Mohamad Alameddine, Beirut (LB); Mohan M.K. Swami, Kuala Lumpur (MY)

(73) Assignee: Health Invest International Limited, Kuala Lumpur (MY)

(21) Appl. No.: 12/756,533

(22) Filed: Apr. 8, 2010

(51) Int. Cl. G06Q 50/00; G06Q 10/00

(52) U.S. Cl. 705/2; 705/5

(57) ABSTRACT

A global health networking system includes at least one server coupled to a network, a plurality of user interfaces, and a plurality of search applications. The network includes a plurality of databases configured to store information from multiple countries relating to academic studies, healthcare procedures and pharmaceuticals, institutes of higher medical related studies, healthcare providers, corporate subscribers, and individuals. The server is configured to communicate between each database such that a user is provided access to information stored in each database for addressing health related topics. Each interface includes user selectable options and is configured to provide access to the databases. The search applications are configured to search the databases according to search parameters entered by the user, wherein the user's needs are linked to information stored in the databases allowing continuous real time access to medical personnel, medical files, and healthcare related information.

### FIELD OF THE INVENTION

The present invention relates to a system and method for providing a global virtual medical city for global health networking. In particular, the present invention relates to a system and method promoting global medical tourism and medical group tours.

### BACKGROUND OF THE INVENTION

In previous years, the now rapidly-growing practice, known as medical tourism (also referred to as medical travel, health tourism, and global healthcare) referred to physicians who traveled or relocated either domestically or internationally to deliver healthcare. In recent years, however, many patients have found that traveling across international borders to obtain healthcare is advantageous for several reasons.

With increasing cost of healthcare, the lack of cooperation by insurance companies to cover certain types of procedures, or the imposition of unreasonable restrictions by insurance companies (e.g., choice of medical facility, location of facility, choice of physician/surgeon, etc.), some patients turn to alternative means for receiving medical care. With the affordability of international travel, it can be desirable to travel to countries offering healthcare at a fraction of the cost of domestic healthcare, particularly if there are minimal difficulties associated with the medical procedure such that international travel to and from the procedure would not be too burdensome (e.g., cosmetic procedures). Further, with the increase of resources available for perusal (e.g., via the internet), knowledgeable, well-calculated decisions can be made regarding specializing physicians and well-known locations/destinations for medical procedures.

Known popular worldwide destinations for medical travel include: Argentina, Bolivia, Belgium, Brunei, Brazil, Cuba, Colombia, Costa Rica, Hong Kong, Hungary, India, Jordan, Lithuania, Malaysia, Mexico, New Zealand, The Philippines, Poland, Saudi Arabia, Singapore, Slovakia, South Africa, South Korea, Thailand, Tunisia, Turkey, UAE, United States. South America, specifically is a popular destination for cosmetic surgery (i.e., plastic surgery) because such procedures in that particular area of the world are commonplace. Countries such as Argentina, Bolivia, Brazil and Colombia have become popular destinations for cosmetic surgery due to the prevalence of skilled and experienced plastic surgeons.

Moreover, with the lack of local healthcare options in combination with long wait times for certain procedures, patient are discovering that waiting extended periods of time for medical care is not necessary.

Currently, patients seeking medical procedure abroad can locate health tourism providers, who connect medical tourists with medical facilities abroad. Typically, the patient seeking a medical procedure abroad contacts a medical tourism provider, providing them with any required information. If the procedure is a necessary procedure, information such as, a medical report, medical history, and diagnosis may be required. The patient must provide the medical information to the health tourism provider, and the provider then passes the information on to appropriate medical facilities abroad. If the procedure is elective and not of necessity, such as certain cosmetic procedures (i.e., breast augmentation, liposuction, tummy tucks, face lifts, etc.), only imperative medical information would need to be provided. The medical tourism provider and patient would then discuss topics including desired cost/budget, choice of hospitals, desired tourist destinations, duration of stay, etc. The provider would ultimately schedule the procedure at the desired hospital and the accommodations at a desired lodging facility.

This process, however, can become inefficient, time consuming, and insecure. Furthermore, because the provider charges a fee for the service, additional costs accrue for the patient. Moreover, current systems do not provide the patient immediate access to medical information relating to credentials of domestic and/or foreign physicians.

Thus, a need exists for a system that allows a user to efficiently plan a medical tour, that is configured to develop, plan and manage customized medical tours such that a patient has immediate access to a plethora of medical information. A need also exists for a system that minimizes manual data entry and data transfers between various systems used to develop a customized medical tour. A further need exists for a system that permits a user to make a medical tour reservation for an entire group.

### SUMMARY OF THE INVENTION

Systems and methods for global health networking and medical group tours are disclosed herein.

One aspect of exemplary embodiments of the disclosed system involves a global health networking system. The system includes a global communications network, at least one server coupled to the network, a plurality of user interfaces, and a plurality of search applications. The network includes a plurality of databases configured to store information from multiple countries relating to academic studies, healthcare procedures and pharmaceuticals, institutes of higher medical related studies, healthcare providers, corporate subscribers, and individuals. The at least one server is configured to communicate between each database from the plurality of databases such that users in the global communications network are provided access to information stored in each database for addressing health related topics; the users being patients, healthcare providers, medical students, medical academics, medical device companies, or pharmaceutical companies. Each interface from the plurality of interfaces includes user selectable options and is configured to provide access to the databases. The search applications are configured to search the databases according to search parameters entered by the user, wherein the user's needs are linked to information stored in the databases allowing continuous real time access to medical personnel, medical files, and healthcare related information.

Another aspect of exemplary embodiments of the disclosed system involves a system for providing medical related services for a global healthcare network. The system includes a plurality of databases, a plurality of applications. The databases include at least a healthcare provider database, a corporate subscriber database, and a patient database, each containing information from multiple regions from around the world. Each database includes a plurality of data sets including at least electronic medical records, appointments, retail/products, employee recruitment, medical training, healthcare publications, academic medical centers, public hospitals, private hospitals, specialized medical clinics, physicians, pharmacies, health product companies, medical technicians, lodging, transportation, job-seekers, and individuals. At least one application from the plurality of applications is configured to link the needs of a user with two or more data sets.

Another aspect of exemplary embodiments of the disclosed system involves a medical tour group system for reserving a medical tour. The system includes a plurality of databases, a medical search application, and a plurality of accommodation search applications. The databases include at least a healthcare provider database, a corporate subscriber database, and a patient database. The medical search application is configured to access information regarding medical services from the healthcare provider database according to search parameters provided by a user and to identify medical facilities offering medical services. The accommodations search applications are configured to access information from the corporate subscriber database regarding lodging accommodations and transportation accommodations. The user can create a customized medical tour according to information provided such that the medical tour includes a desired medical procedure, a desired medical facility, desired transportation accommodations, and desired lodging.

Another aspect of exemplary embodiments of the disclosed system involves a method for reserving a medical tour. User profile information is first entered into a patient database of the medical tour group system. The information includes specific medical and accommodations preferences. A medical service request is submitted to the medical tour group system. A medical search application of the system accesses information regarding medical services from a plurality of databases according to the medical service request and user specific medical preferences. An accommodations request is submitted to the medical tour group system. A plurality of accommodation search applications access information from the plurality of databases according to the accommodations request and user specific accommodations preferences.

### BRIEF DESCRIPTION OF THE DRAWINGS

The exemplary embodiments of the disclosed system can be better understood with reference to the following drawings and description. The components in the figures are not necessarily to scale, emphasis instead being placed upon illustrating the principles of exemplary embodiments of the disclosed system. Moreover, in the figures, like reference numerals designate corresponding parts through the different views.

FIG. 1 is a block diagram illustrating an electronic global health networking system according to an embodiment of exemplary embodiments of the disclosed system.

FIG. 2 illustrates the components of a server of the electronic global health networking system of FIG. 1.

FIG. 3 illustrates network connections of an electronic global health networking system according to an embodiment of exemplary embodiments of the disclosed system.

FIGS. 4A-4R illustrate a process for a medical professional to log on to and maneuver through the health networking system of FIGS. 1 and 2.

FIGS. 5A-5C illustrate a process for a patient to log on and purchase a healthcare package within the health networking system of FIGS. 1 and 2.

FIGS. 6A-6G illustrate a process for a broker to log on and maneuver through the health networking system of FIGS. 1 and 2.

FIGS. 7A-7C illustrate a process for a provider to log on and maneuver through the health networking system of FIGS. 1 and 2.

FIGS. 8A-8I illustrate a process for a receptionist to log on to the health networking system of FIGS. 1 and 2 and schedule appointments for patients.

FIGS. 9A-9D illustrate a process for a user to submit applications to a plurality of facilities via the health networking system of FIGS. 1 and 2.

FIG. 10 illustrates a process for a user to post a CV or resume on the health networking system of FIGS. 1 and 2

FIG. 11 illustrates a process for a user to search directories of the health networking system of FIGS. 1 and 2.

FIGS. 12A-12R illustrate screen shots, corresponding to FIGS. 4A-4R, that might be presented to a medical professional for prompting entry of information to maneuver through the health networking system of FIGS. 1 and 2.

FIGS. 13A-13G illustrate screen shots, corresponding to FIGS. 5A-5C, that might be presented to a patient for prompting entry of information for purchasing a healthcare package within the networking system of FIGS. 1 and 2.

FIGS. 14A-14I illustrate screen shots, corresponding to FIGS. 6A-6G, that might be presented to a broker for prompting entry of information to maneuver through the health networking system of FIGS. 1 and 2.

FIGS. 15A-15C illustrate screen shots, corresponding to FIGS. 7A-7C, that might be presented to a provider for prompting entry of information to maneuver through the health networking system of FIGS. 1 and 2.

FIGS. 16A-16G illustrate screen shots, corresponding to FIGS. 8A-8I, that might be presented to a receptionist for prompting entry of information for assisting patients on the health networking system of FIGS. 1 and 2.

FIGS. 17A-17D illustrate screen shots, corresponding to FIGS. 9A-9D, that might be presented to a user when submitting an application on the health networking system of FIGS. 1 and 2.

FIGS. 18A and 18B illustrate screen shots, corresponding to FIG. 10, that might be presented to a user when posting a curriculum vitae or resume on the health networking system of FIGS. 1 and 2.

FIG. 19 illustrates a screen shot, corresponding to FIG. 11, that might be presented to a user when searching directories of the health networking system of FIGS. 1 and 2.

FIG. 20 is a flow chart illustrating a method for reserving a medical tour or medical tour group using an electronic group tour system according to an embodiment of exemplary embodiments of the disclosed system.

### DETAILED DESCRIPTION OF THE DRAWINGS

The present invention includes a system for networking within the healthcare arena as well as for organizing and scheduling a medical tour. The system includes an interactive global network community wherein the network allows participants continuous real time access to medical personnel, medical files, and healthcare related information.

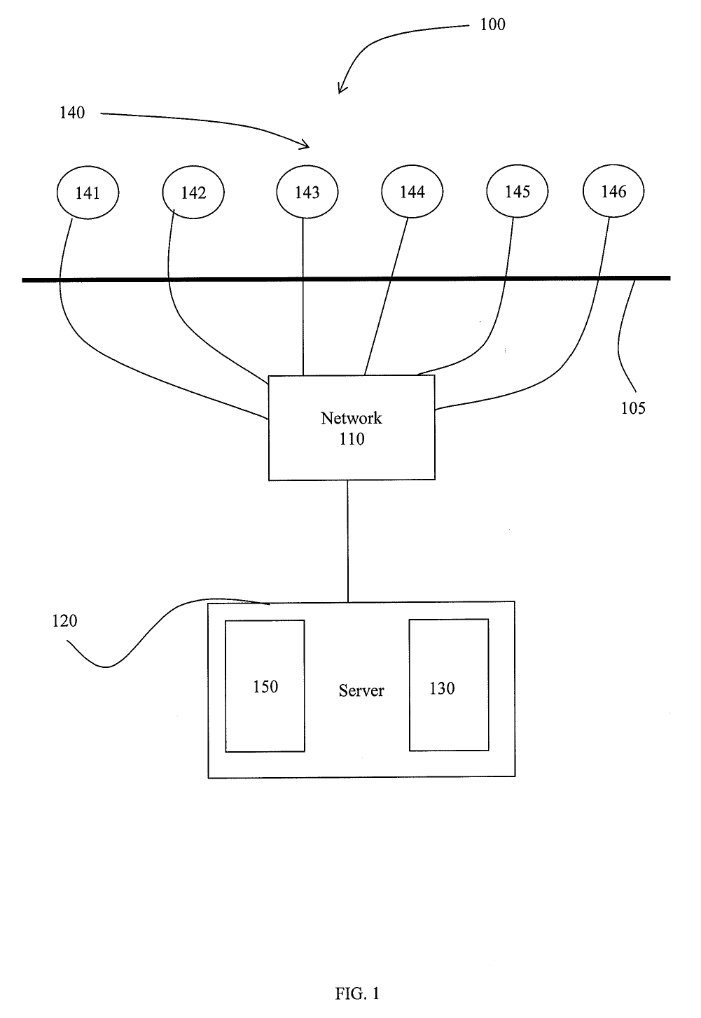

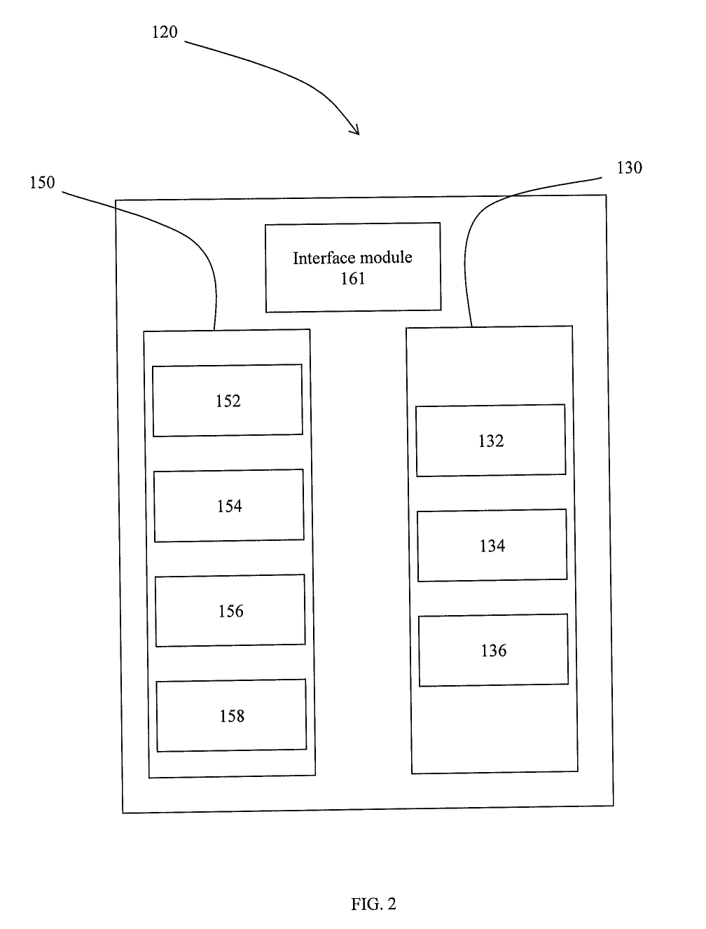

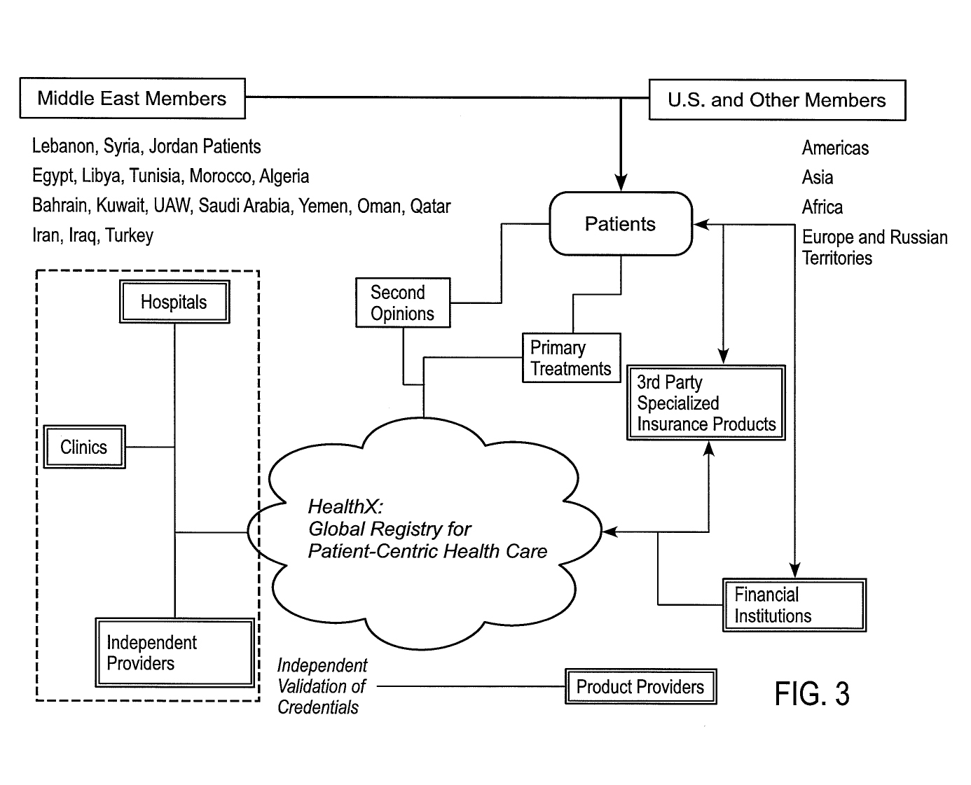

FIGS. 1 and 2 illustrate an electronic global health networking system 100 according to an embodiment of the disclosed system. Referring to FIG. 1, the system 100 includes a global communications network 110 (referred to herein as “network”), at least one server 120 (referred to herein as “server”), and a plurality of user interfaces 140.

The network 110 is coupled to the server 120 and to the plurality of user interfaces 140 and is configured to allow communication between applications and/or devices. The network 110 can include any software and hardware device used to provide communication between applications/devices and can include any design configured to enable transfer data (e.g., the Internet). Communication within the network 110 can be performed via a wired or wireless based communication and can be any communication medium configured to transmit data.

The server 120 is coupled to the network 110 and includes at least one processor and at least one memory device. The server further includes a plurality of databases 130 and an application system 150, each stored within the memory device of the server 120, and at least one communication interface module 161.

The plurality of databases 130 is configured to store a variety of information gathered from multiple countries from around the world. The application system 150 includes a plurality of search applications 152-158 configured to search the plurality of databases 130. The plurality of search applications 152-158 is configured to be executed by the server 120 and to search the plurality of databases 130 according to search parameters entered by a user (described in more detail herein).

The plurality of databases 130, as illustrated in FIG. 2, includes at least a corporate subscriber database 132, a healthcare provider database 134, and a patient database 136 and is configured to store information relating to, for example, academic studies, healthcare procedures, pharmaceuticals, institutes of higher medical related studies, healthcare providers, corporate subscribers (e.g., hotels, motels, bed and breakfasts, resorts, vendors for popular trip items, salons/spas, financial institutions, etc.), and individuals (e.g., patients, physicians, pharmacists, job seekers, etc.). More specifically, the databases are configured to receive and store information provided by authorized users. For example, the patient information can include any information provided by the patient (e.g., contact information, electronic medical records, etc.). The physician information can include, for example, contact information, areas of specialty, resume/credential, publications, speaking engagements, patents, etc. The corporate subscribers information can include, for example, hotel and transportation accommodation information. The hotel accommodation information can include, for example, hotel pricing, amenities, group pricing, etc. The transportation accommodation information can include, for example, transportation pricing for a variety of methods of transportation (e.g., airfare, bus, train, etc.), group pricing, etc. Said another way, authorized users of the system 100 can add and/or edit account information within the plurality of databases 130.

In some embodiments, the server 120 can include a database management system (not illustrated) configured to control the creation, organization, maintenance, and the use of the plurality of databases 130. In such embodiments, a user can store and retrieve data in an extremely structured manner. For example, a user can submit a request to the database management system for information regarding a desired medical procedure. The database management system accepts the request, locates the desired data from the healthcare provider database 134, and instructs the operating system to transfer the appropriate data from the database to the user interface.

As illustrated in FIG. 2, the application system 150 includes a plurality of search applications 152-158 and is configured to provide the functionality of the global health networking system 100. More specifically, the application system 150 is configured to search the plurality of databases 130 according to search parameters entered by the user (described in more detail herein) and receive information from the plurality of databases 130 to provide it to the user. The search applications 152-158 include at least, for example, a medical search application 152, an accommodation search application 154, patient search application 156, and a reservation application 158. The medical search application 152 is configured to access and search databases associated with medical information (e.g., information relating to physicians, medical facilities, medical procedures, medical related studies, etc.). The accommodation search application 154 is configured to access and search databases associated with accommodation information (e.g., lodging and transportation accommodations). The patient search application 156 is configured to access and search the patient database such that patient information can be retrieved. The reservation application 158 is configured to reserve a medical procedure, lodging and transportation accommodations, as well as other request, according to user instructions.

Although the application system 150 is described as including the abovementioned search applications, it should be understood that the application system 150 can include any suitable number of search applications. For example, in some embodiments, the application system can include a financial search application. In other embodiments, the application system can further include a pharmaceutical search application. In yet other embodiments, the application system can further include a medical device search application.

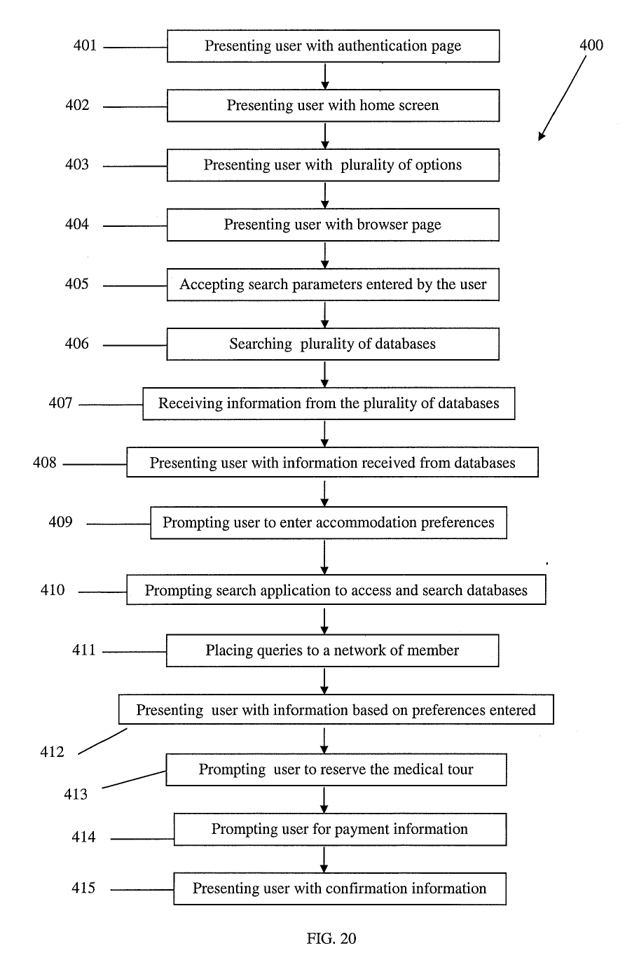

The at least one communication interface module 161 is configured to provide access to the application system 150 and the plurality of databases 130 such that a user can communicate with the plurality of databases 130 to enter or delete information. Said another way, the communication interface module 161 provides a communication path for a user to review, update, and/or delete their own personal information stored in the databases 130. The at least one communication interface module 161 includes at least one security module (not illustrated) configured to allow an authorized user access to the global health network by verifying identification information provided by the user. The security module is described in more detail herein with reference to FIG. 20.

In some embodiments, the server 120 can include a plurality of communication interface modules (not illustrated). In such embodiments, each interface module from the plurality of modules is configured to provide communication between a corresponding user interface, the plurality of databases, and the application system. Said another way, each user interface device can be configured to communicate with the server 120 via a corresponding network interface. The interface module can include any suitable number of interface modules for providing communication to corresponding user interfaces 140. For example, in some embodiments, the plurality of communication interface modules can include a patient interface module corresponding to a patient interface 141. In other embodiments, the plurality of communication interface modules can include a healthcare provider module corresponding to a healthcare provider interface 142 (e.g., physicians, pharmacists, medical technicians, nurses, etc.). In yet another embodiment, the plurality of communication interface modules can include a corporate interface module corresponding to corporate subscribers interface 143 (e.g., insurance providers).

The plurality of user interfaces 140 (mentioned above) (also referred to herein as “user interfaces” and “interface devices”) is coupled with the network 110 (e.g., is in communication with) and is configured to provide a user access to information stored in the plurality of databases 130 on the server 120. The plurality of interface devices 140 can include any type of device configured to provide a connection for communication over the network 110 and can include a plurality of user selectable options. The interface devices 140, for example, can include a personal computer (PC), a personal digital assistant (PDA), a wireless telephone, a network terminal, a server computer, or any other wireless or wireline devices. The interface devices 140 can include a plurality of visible graphical interface features (e.g., menus, icons, links, dialog boxes, etc.) and are configured to allow data entry by the user. The interface devices 140 are further configured to launch and operate an internet browser (e.g., Internet Explorer™, Microsoft™, etc.) for communication with the server 120. The browser is configured to locate, display, and present audio and visual information on pages downloaded from the network 110 including, for example, text, graphics, video, and/or multimedia. The interface devices 140 operate via the World Wide Web, which is separated from the network 110 by a security mechanism 105, which permits only authorized users from accessing the network 110. Any suitable security mechanism can be used. For example, in some embodiments, a security firewall can be used.

The user can be any individual or facility associated with medical related fields or interested in health related topics/information. In one embodiment a user can be, for example, patients, medical students, healthcare providers, insurance providers, job seekers, etc. In other embodiments, the user can be, for example medical academic institutions (e.g., medical school, pharmacy school, nursing school, etc.), medical facilities (e.g., public or private hospitals, clinics, rehabilitation centers, etc.), medical device companies, pharmaceutical companies, insurance companies, etc.

In order to maintain secure communication between the server 120 and the plurality of user interfaces 140 of the system, security applications (not illustrated) can be included in the network 110. For example, secure data transmission can involve a secure socket layer (SSL) connection or encryption/decryption capability. Establishment of secure communications may also include user login information including user names, passwords, and personal numerical codes (described in more detail herein).

As depicted in FIG. 1, the plurality of interfaces 140 includes interfaces corresponding to the aforementioned users. For example, the plurality of interfaces 140 can include at least a patient interface 141, a healthcare provider interface 142, an corporate subscriber interface 143, job seeker interface 144, medical facility interface 145, medical university interface 146, etc. Although the plurality of interfaces 140 is described as including the above mentioned interfaces 141-146, it should be understood that any suitable number of interfaces can be used. As mentioned above, each interface from the plurality of interfaces 140 includes user selectable options and is configured to provide access to the plurality of databases 130. The user interfaces 140 are configured to allow authorized users (described in more detail herein) access to the global health network.

As previously mentioned, the security module is responsible for the verification of log in information provided by the user. Because the global health networking system 100 includes a plurality of different access levels, authorized users are permitted access to capabilities corresponding to the level of access to which they have been assigned. For example, a patient gaining access to the server via the patient interface 141 may have access to his/her electronic medical records and contact information. The patient could update or delete his/her medical files and could also schedule an appointment with a physician associated with the network 110. The patient would also be permitted to search the plurality of databases 130 to view physician records and credentials, medical facilities, insurance providers, hotel and transportation accommodations, etc. The patient, however, would not have access to another patient's medical files or financial files. Similarly, a physician gaining access to the server 110 via the healthcare provider 142 interface would have access, for example, to his/her own personal files. The physician could update or change his/her contact information, area of specialty, hospital affiliation, publications and/or speeches. The physician would not have access to a patient's medical records unless he/she receives patient authorization. In such a case, because of the various access levels and added security, the system 100 will prompt the physician to enter additional security information. For example, each authorized user of the global health network 110 is assigned a personal numerical code, specific to each user. In cases of added security, the global health networking system 100 will prompt the user for entry of a personal security code. As in the example above, the physician would be required to enter the personal numerical code of the patient whose medical records the physician would like to view. In that case, the patient must provide the physician with the code in order for the physician to access and view the files.

Once the user has been granted access to the global health networking system 100, the system 100 then presents the user with a plurality of choices and/or options. As described earlier, user interfaces 140 of the system 100 are configured to allow data entry by the user and includes a plurality of visible graphical interface features (e.g., menus, icons, links, dialog boxes, etc.). These features allow the user to maneuver easily within the system 100 so that they can, for example, update their profile information, medical records, and contact information. Other options can include searching the plurality of databases 130 for desired information requested by the user, allowing the user to purchase medical device products and certain pharmaceuticals; permitting the user to reserve transportation accommodations and lodging accommodations (described in more detain herein).

The following examples describe the many different functions of the global health networking system 100 as well as the plurality of different users permitted to access the system 100.

### EXAMPLE 1

### Physician

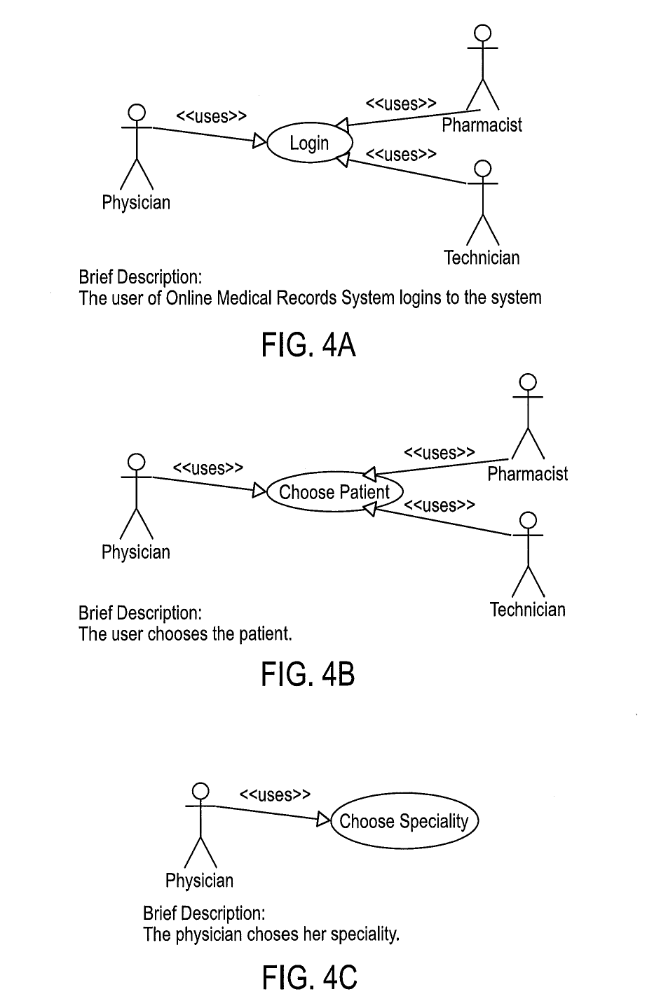

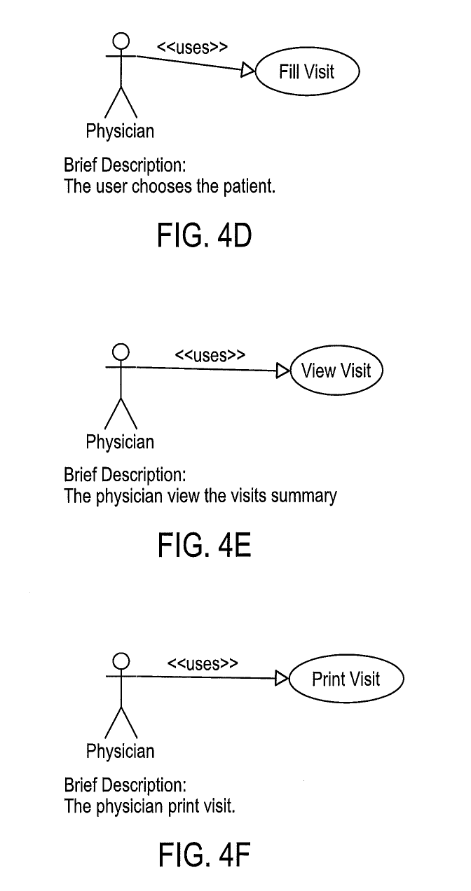

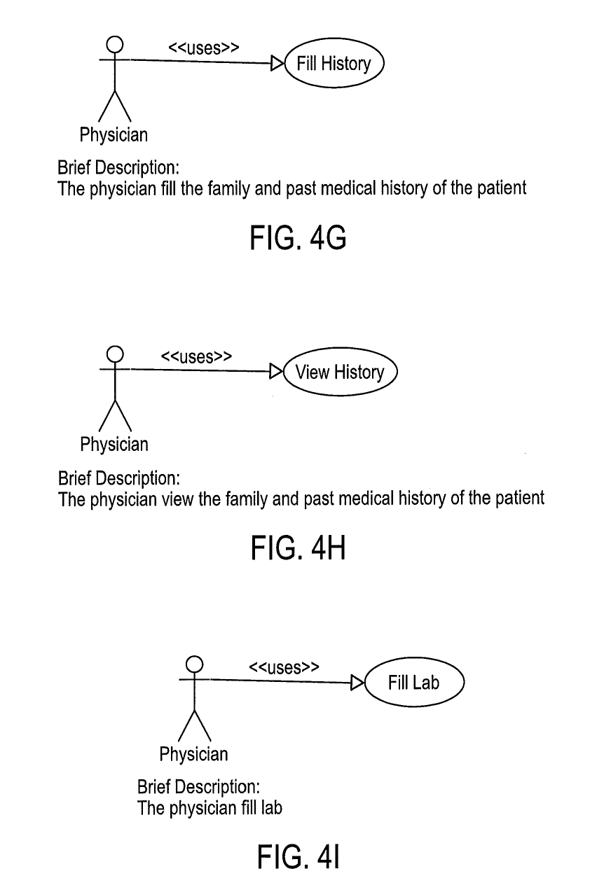

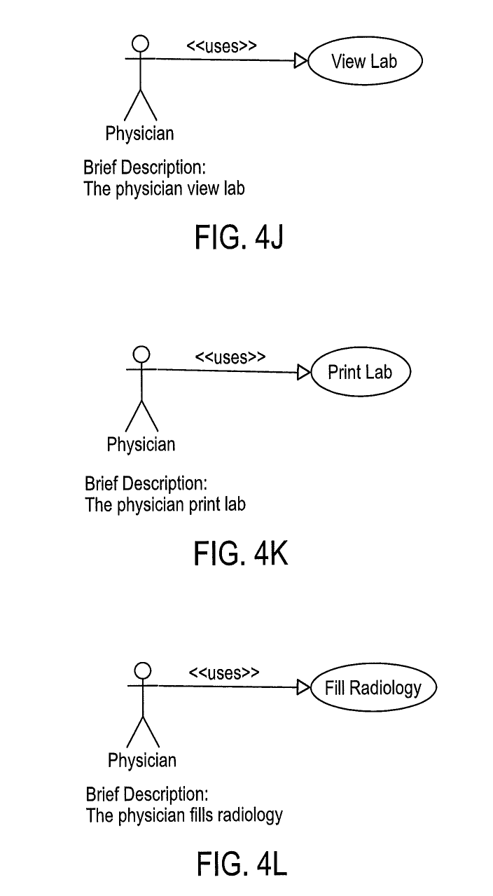

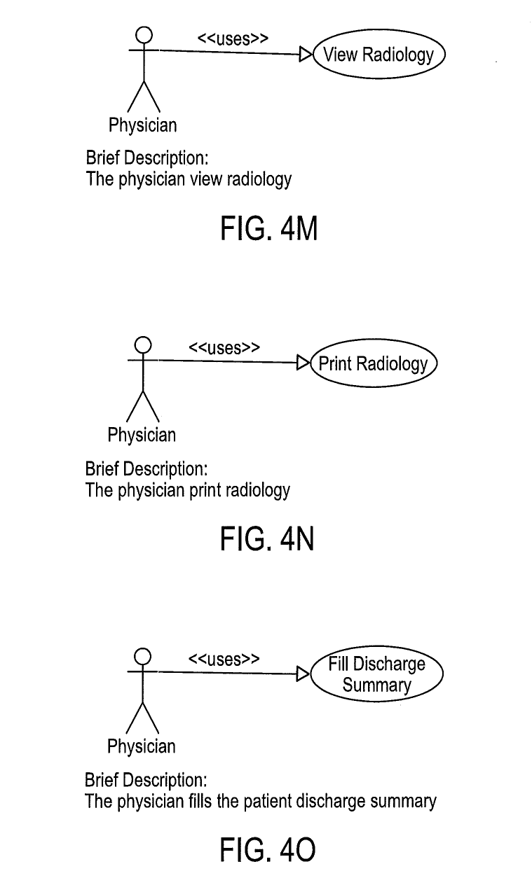

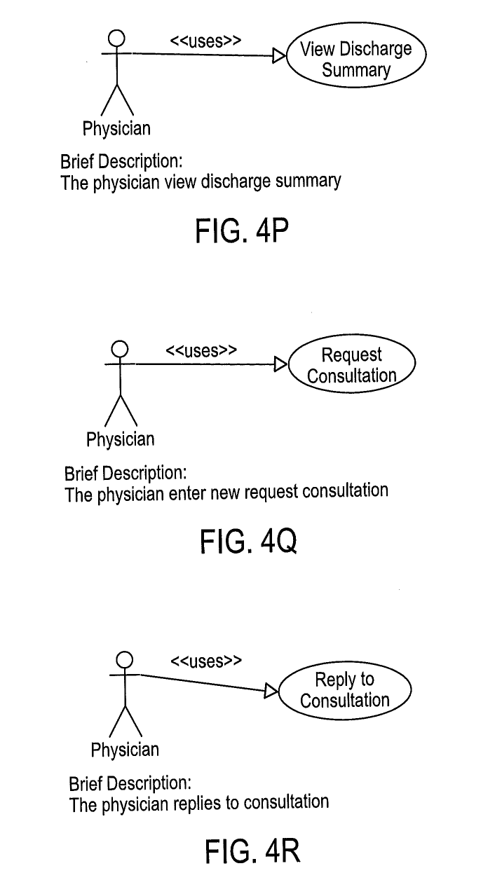

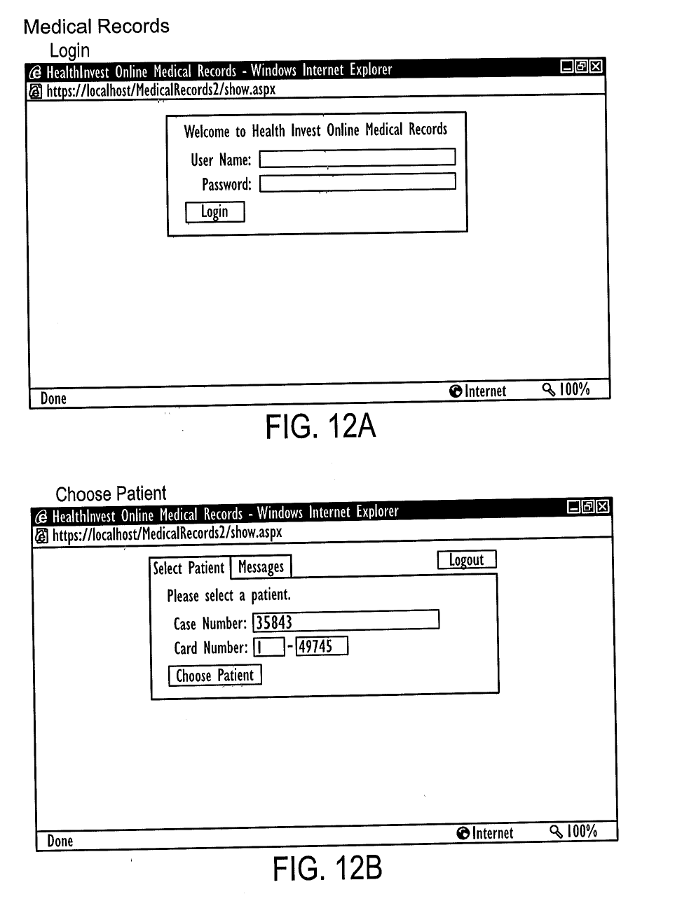

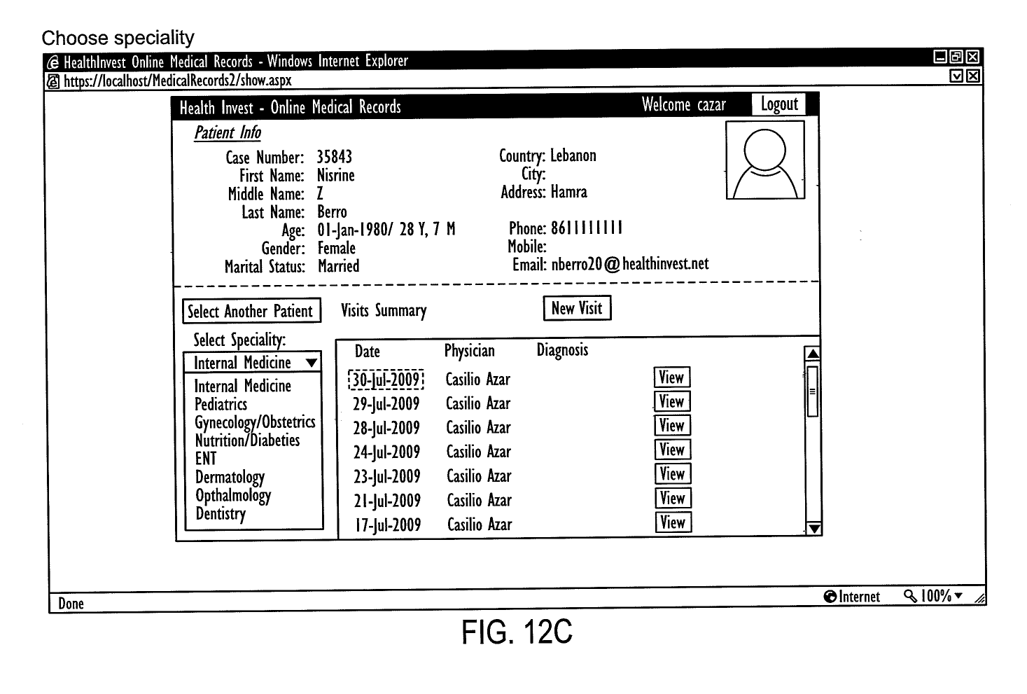

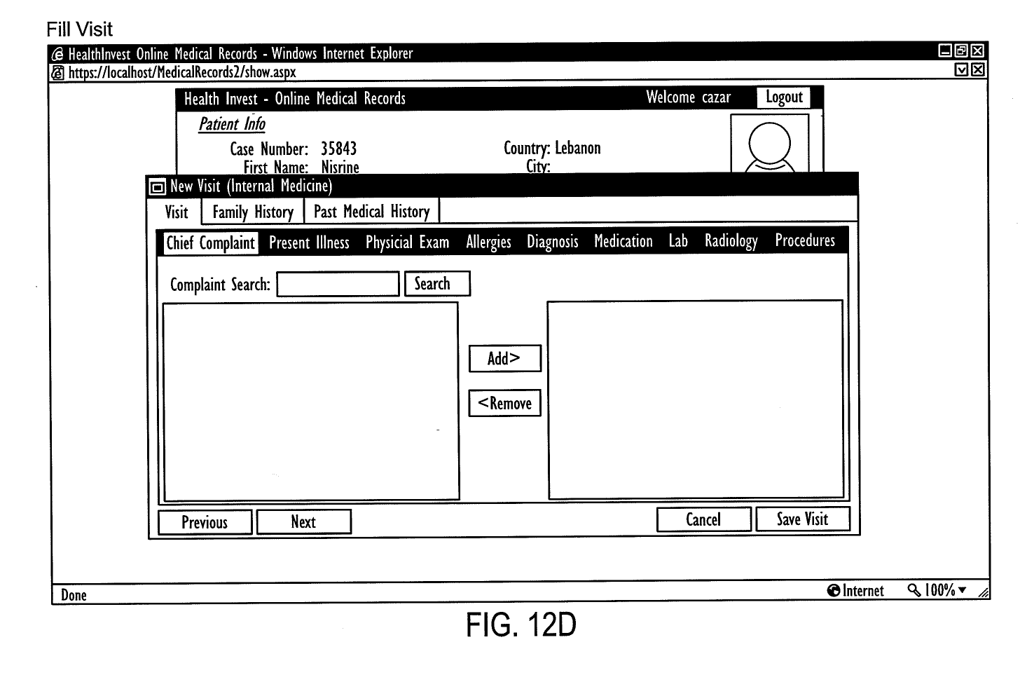

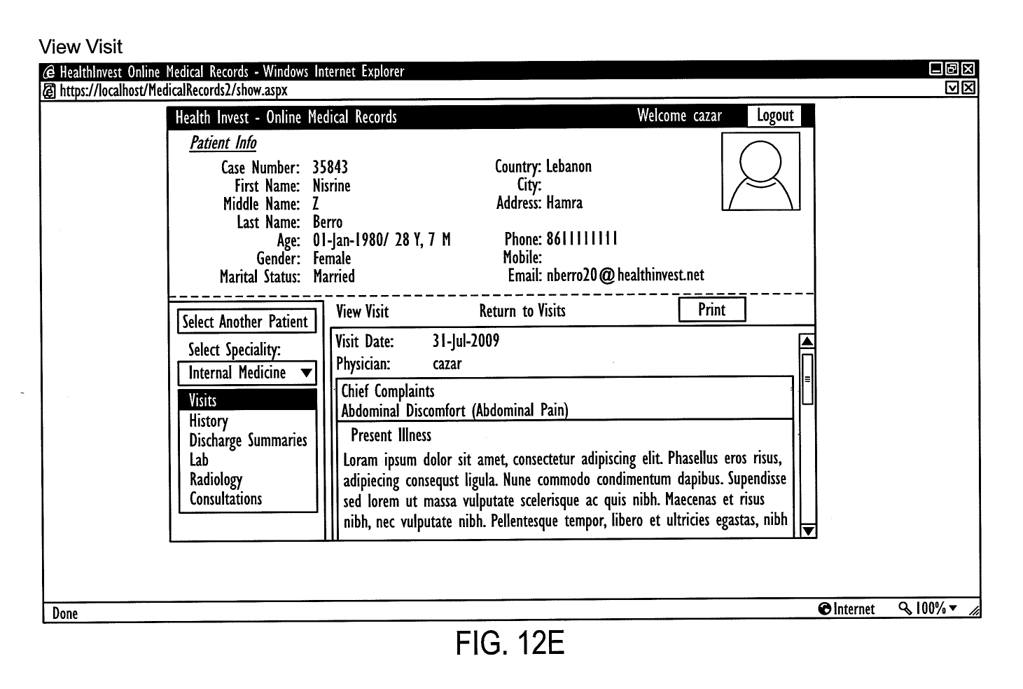

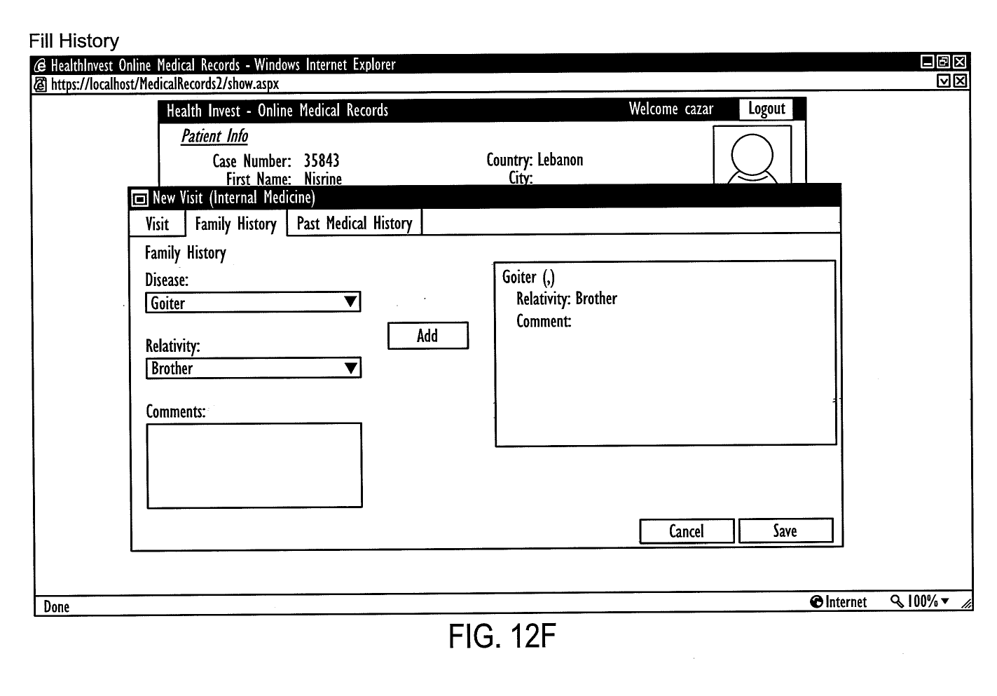

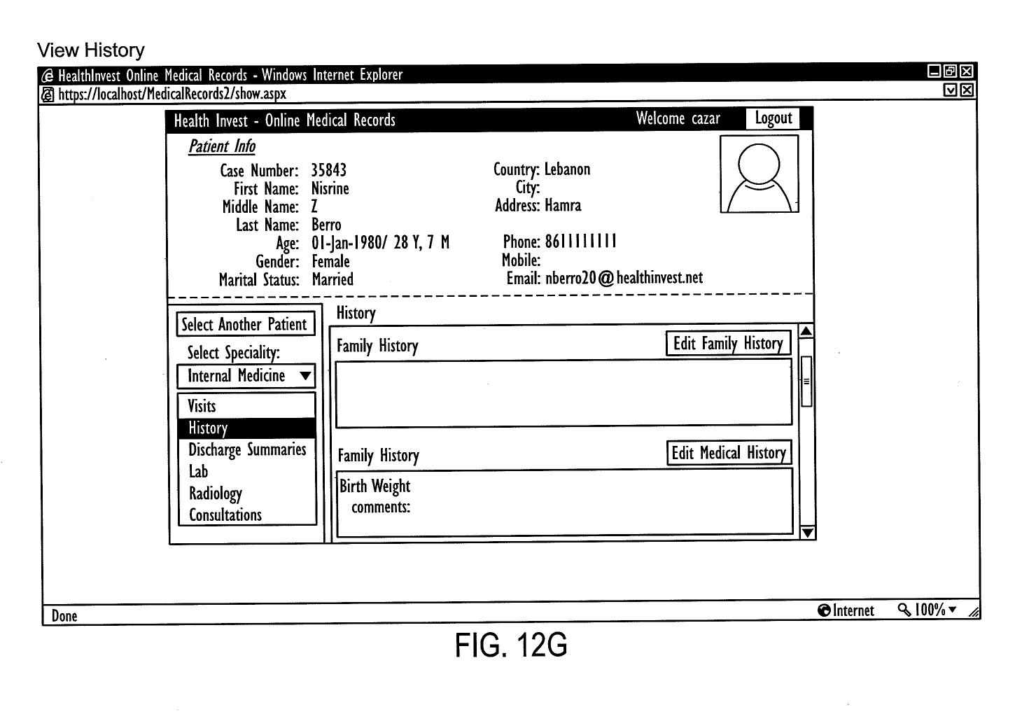

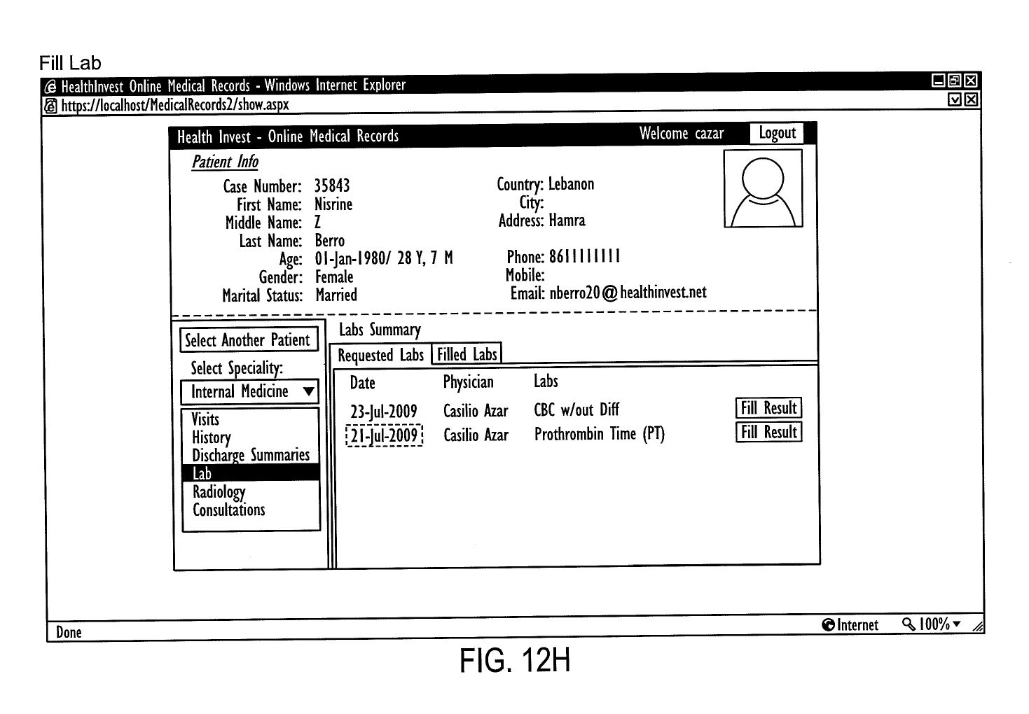

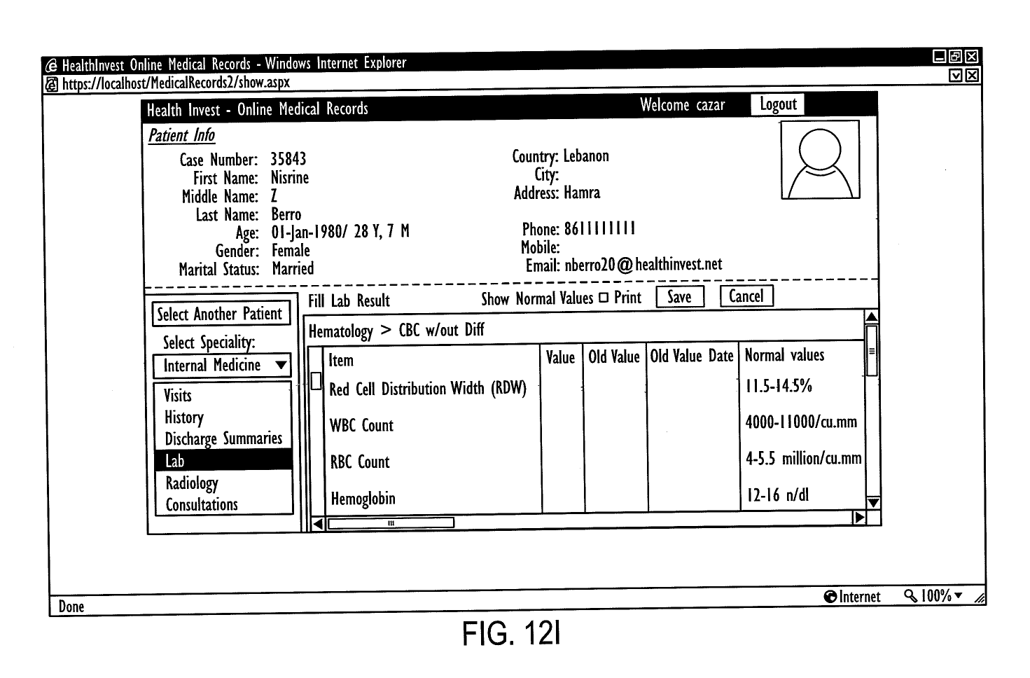

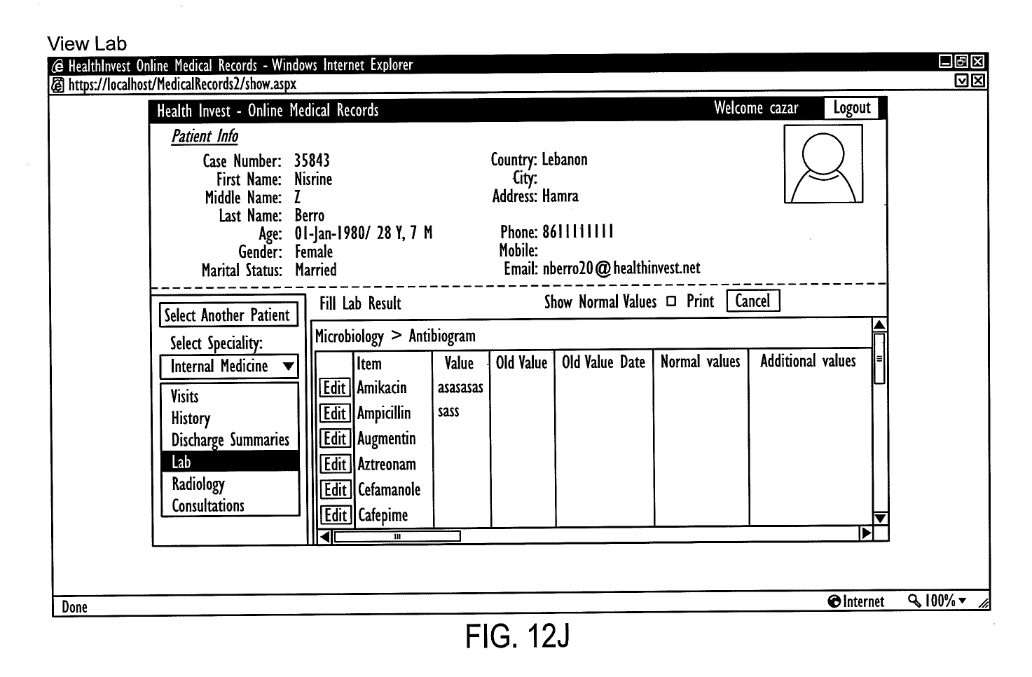

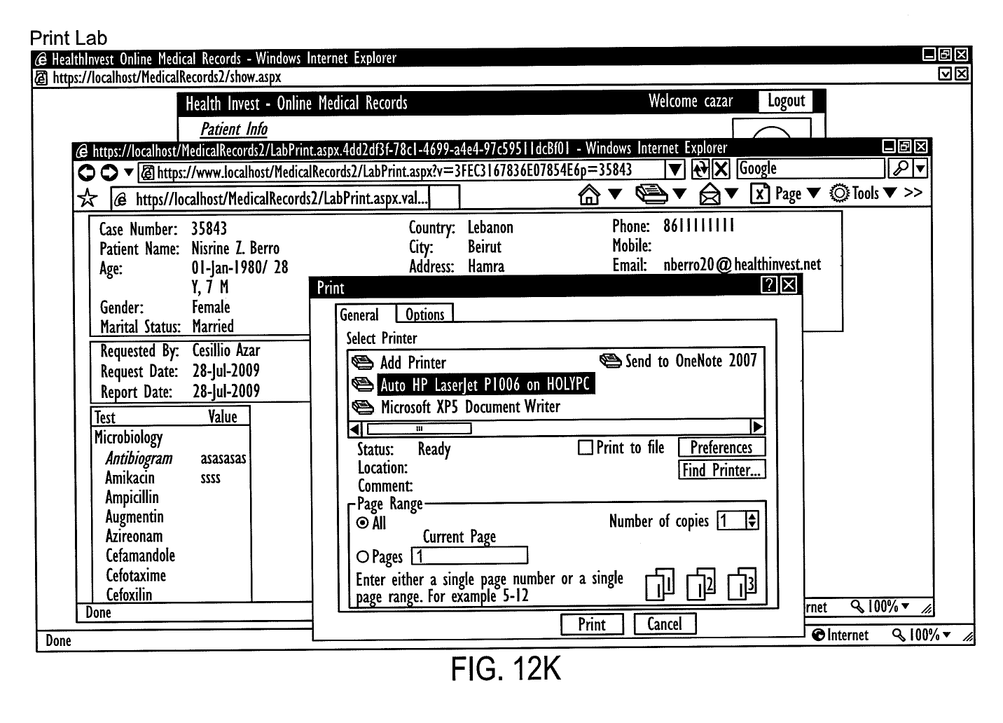

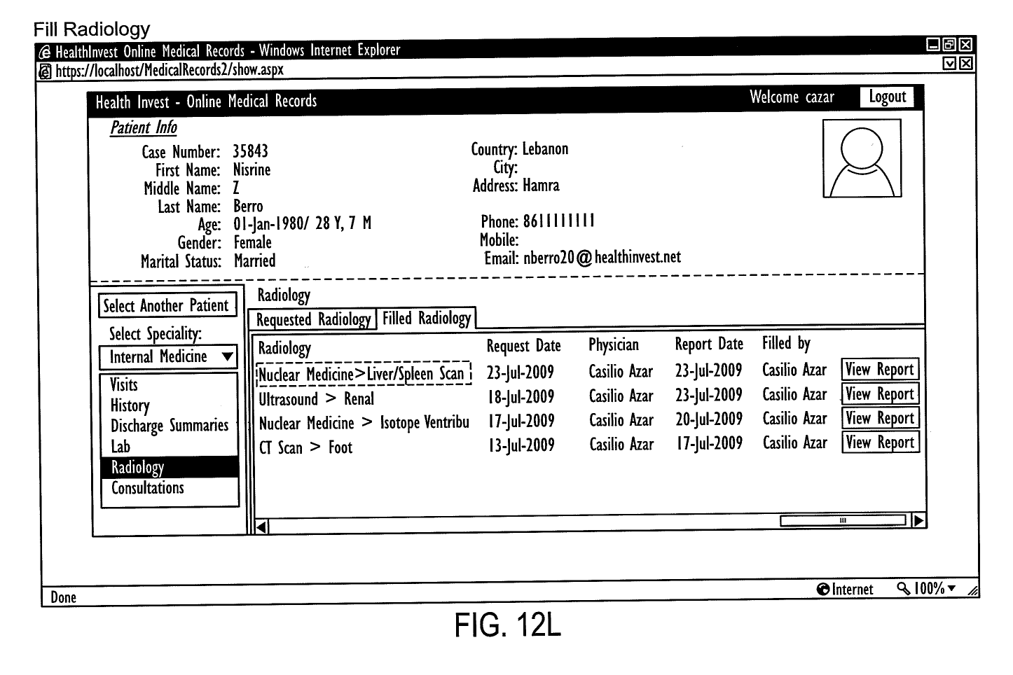

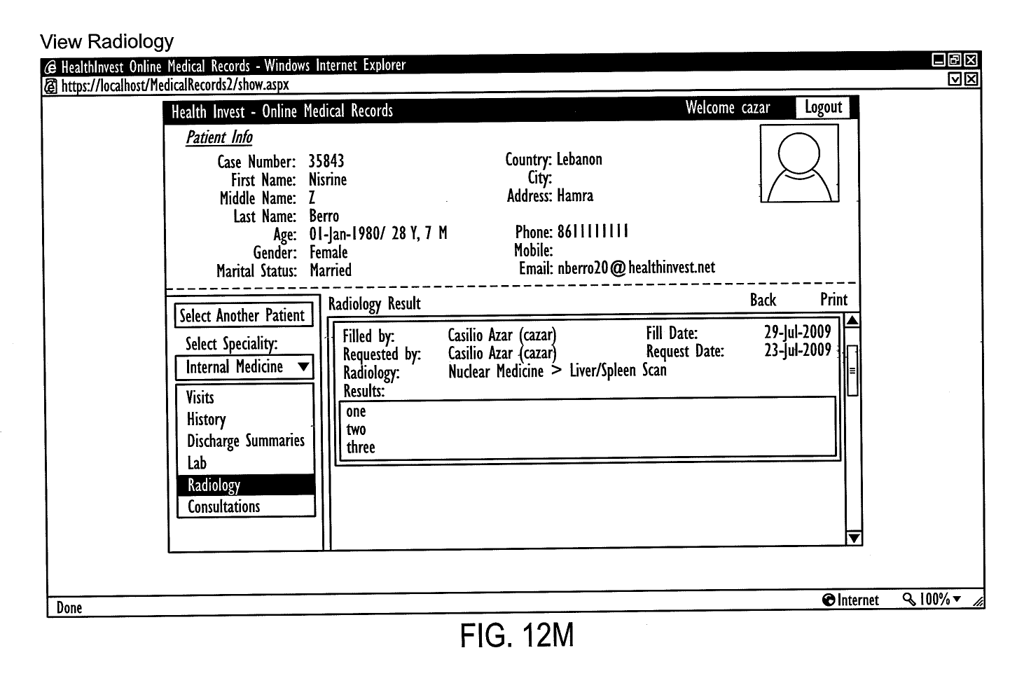

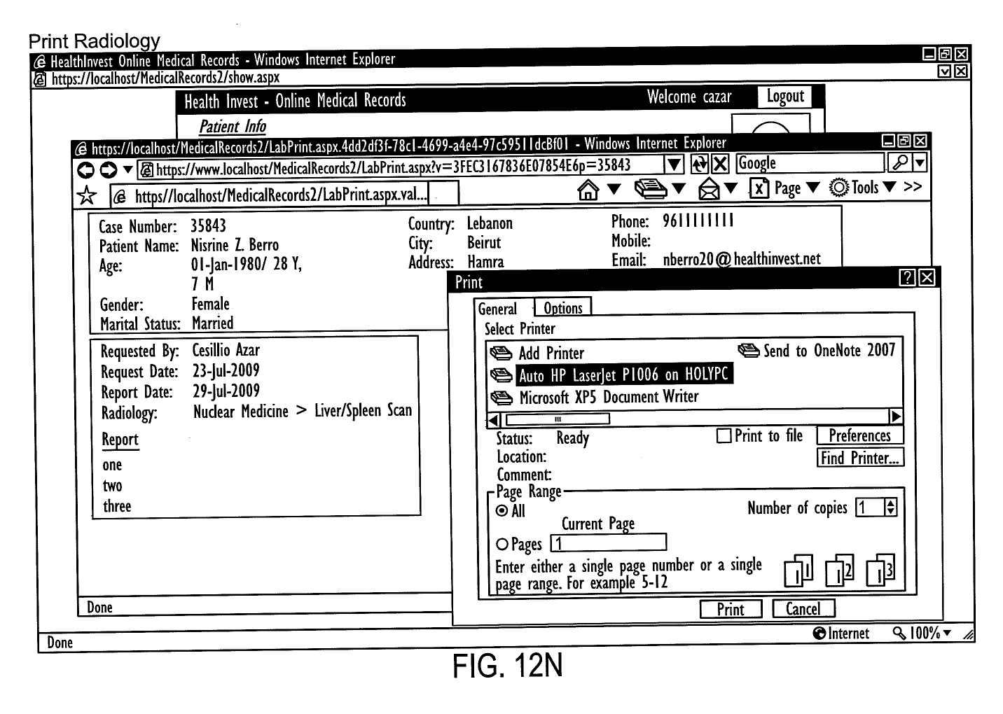

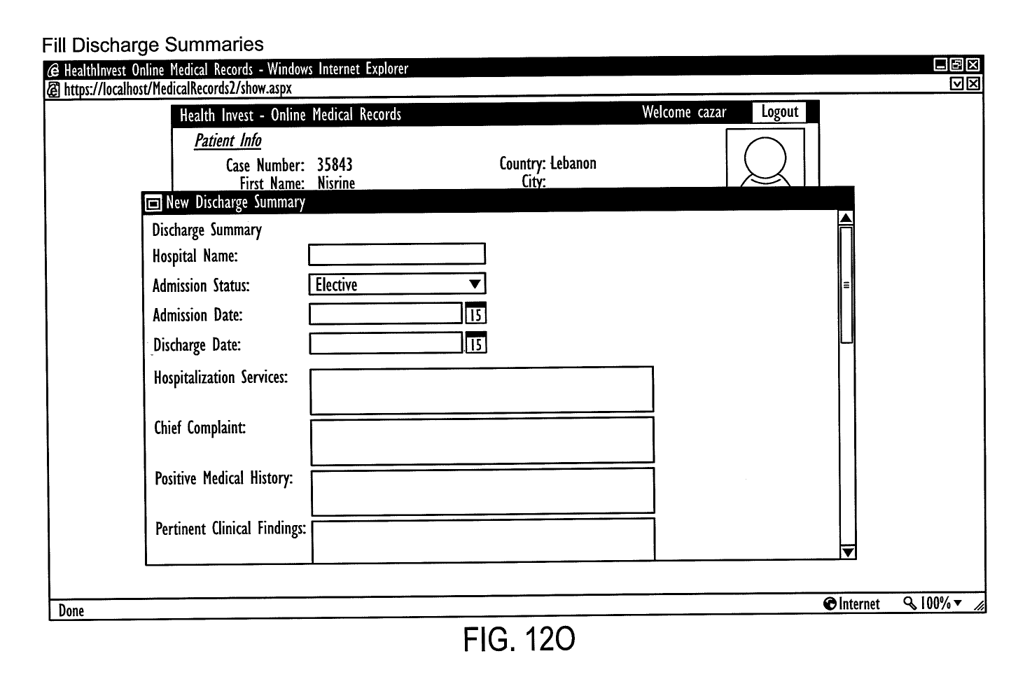

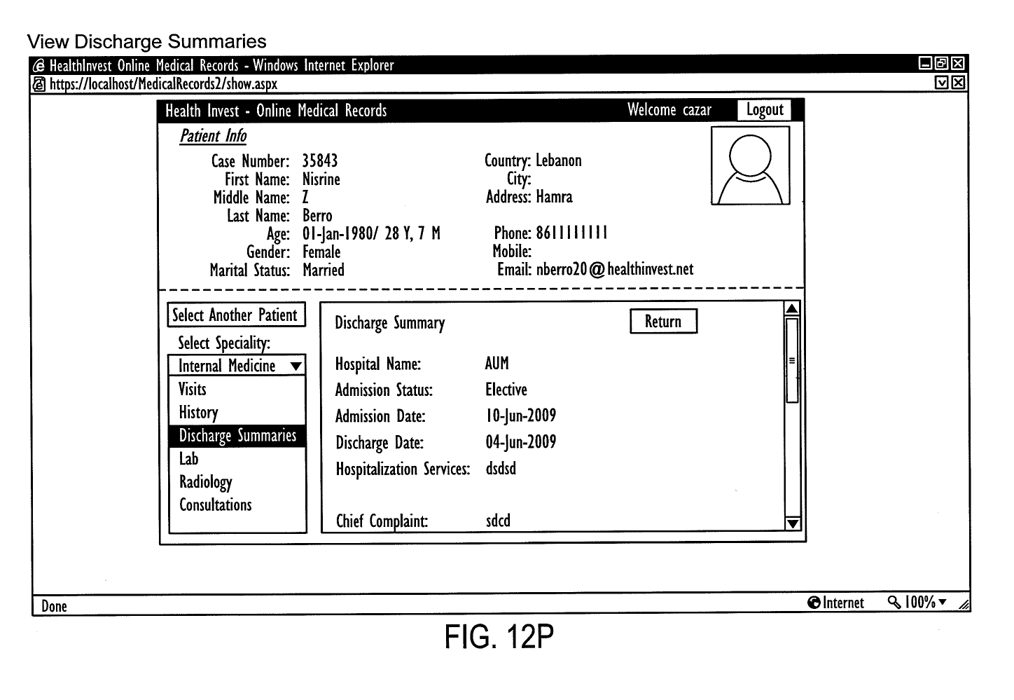

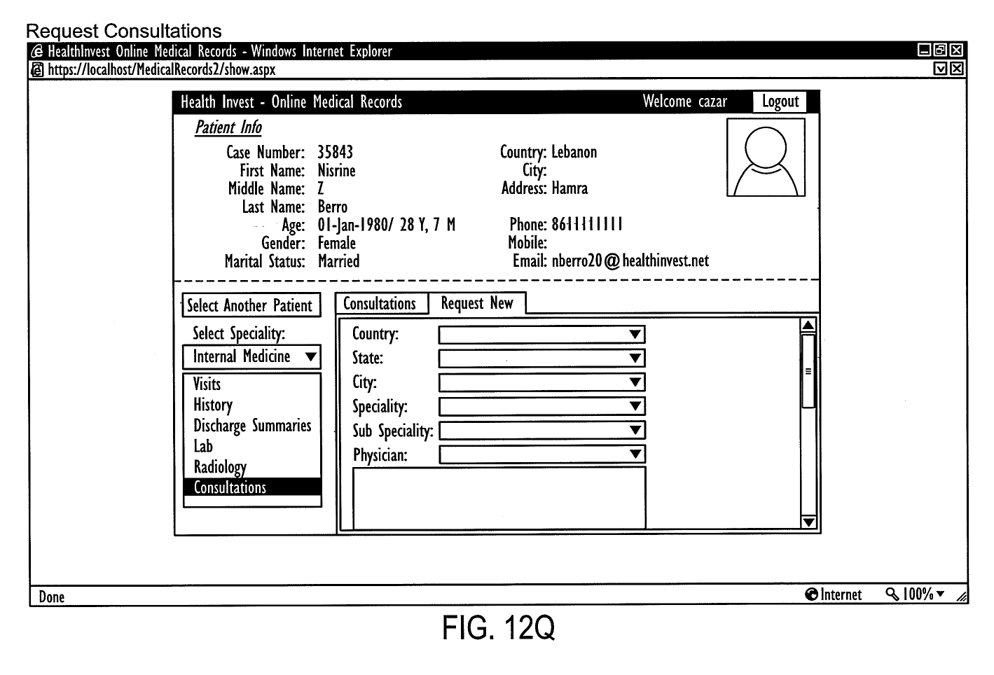

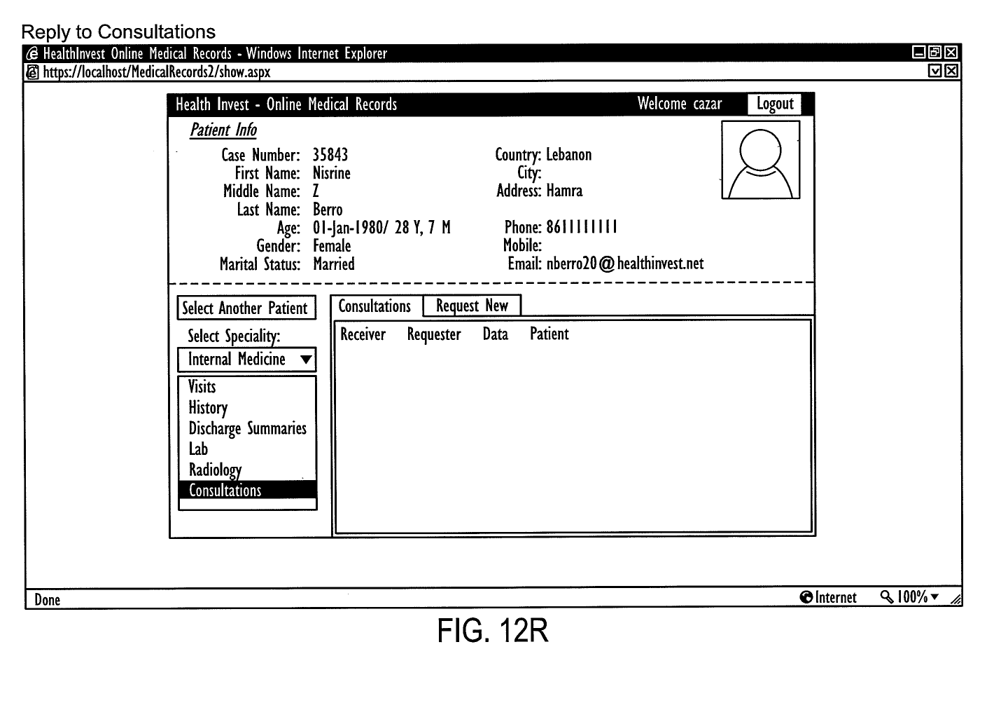

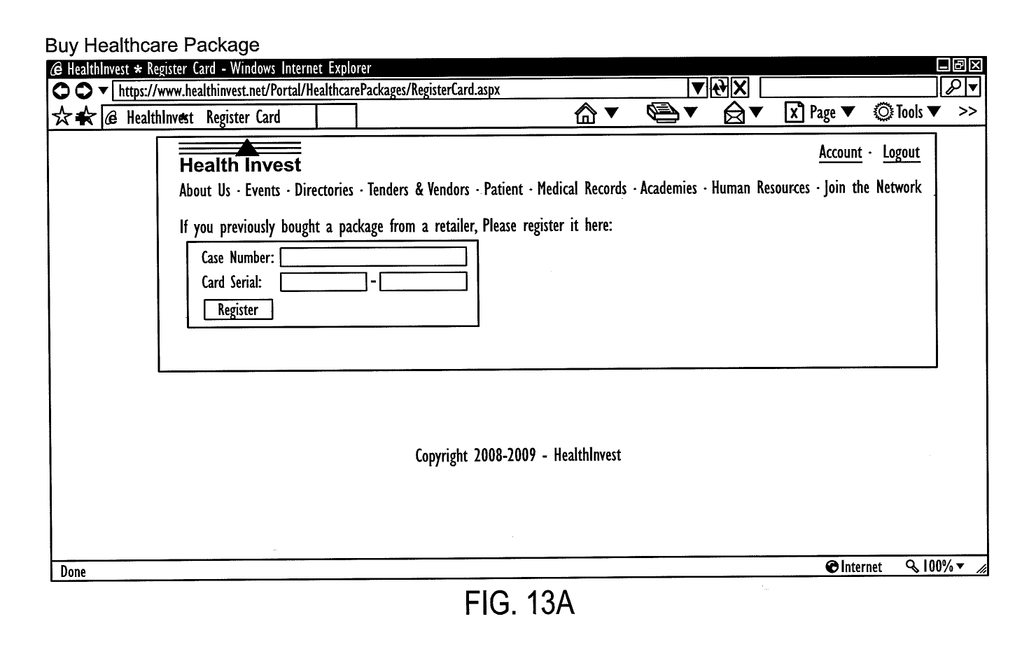

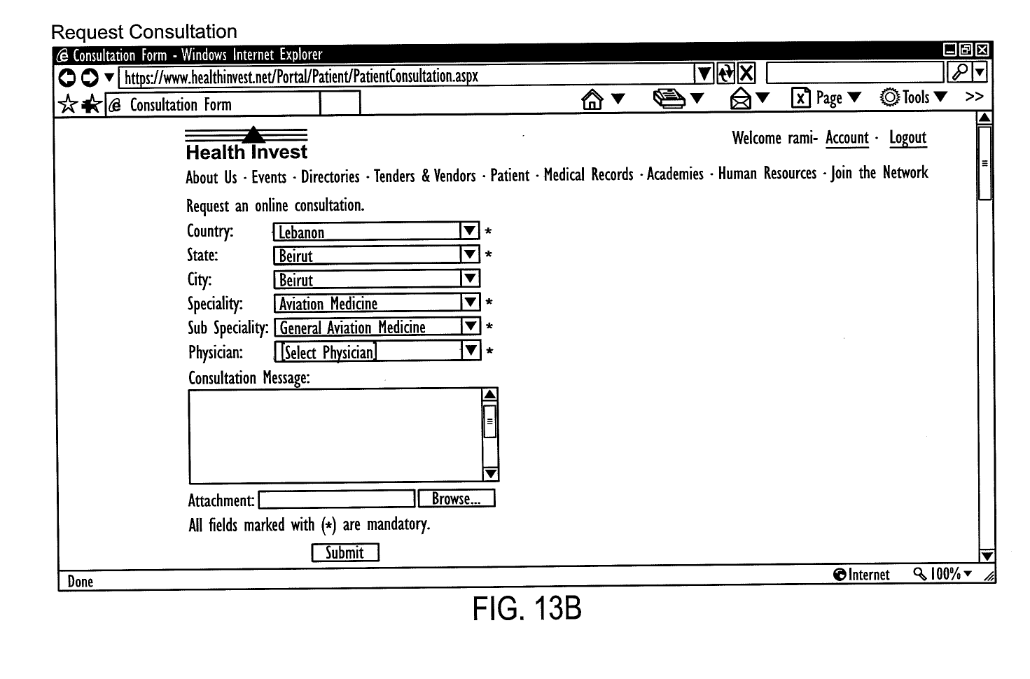

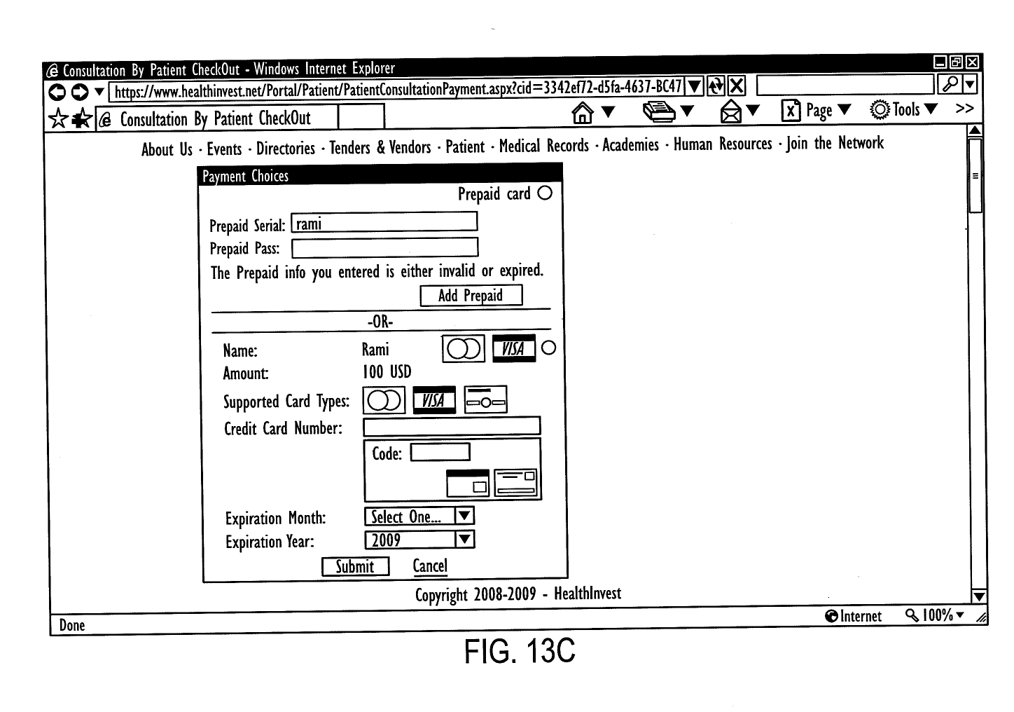

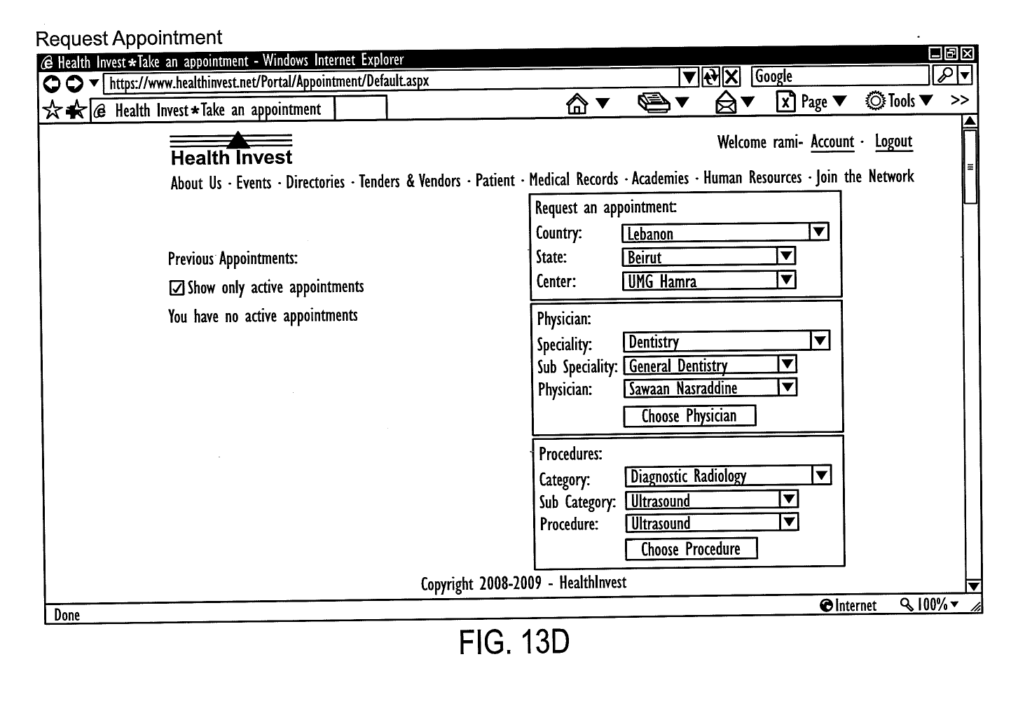

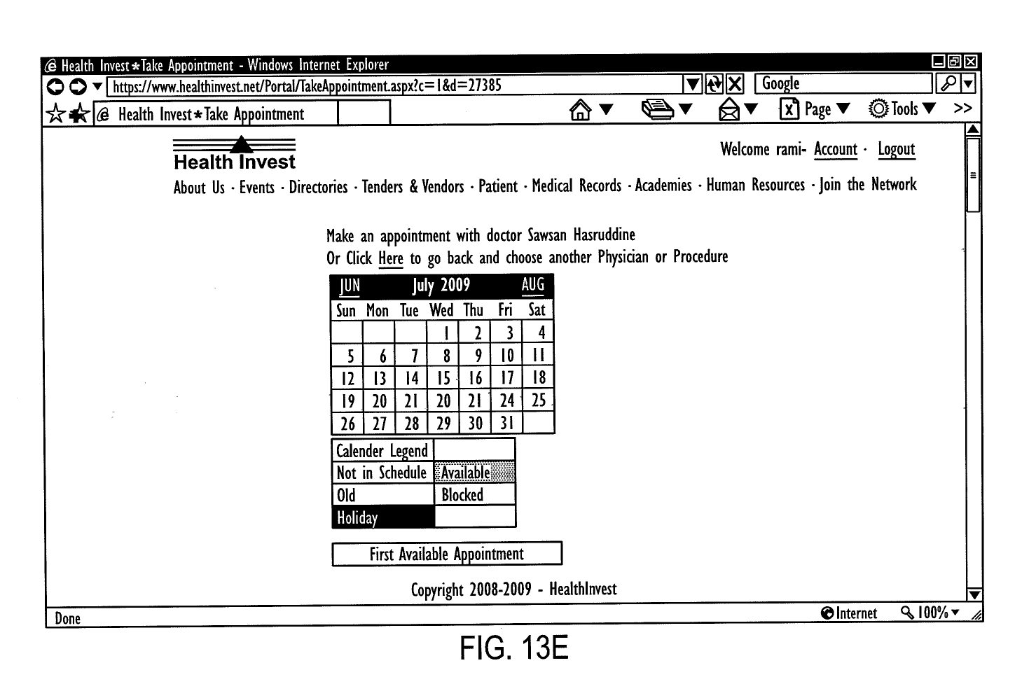

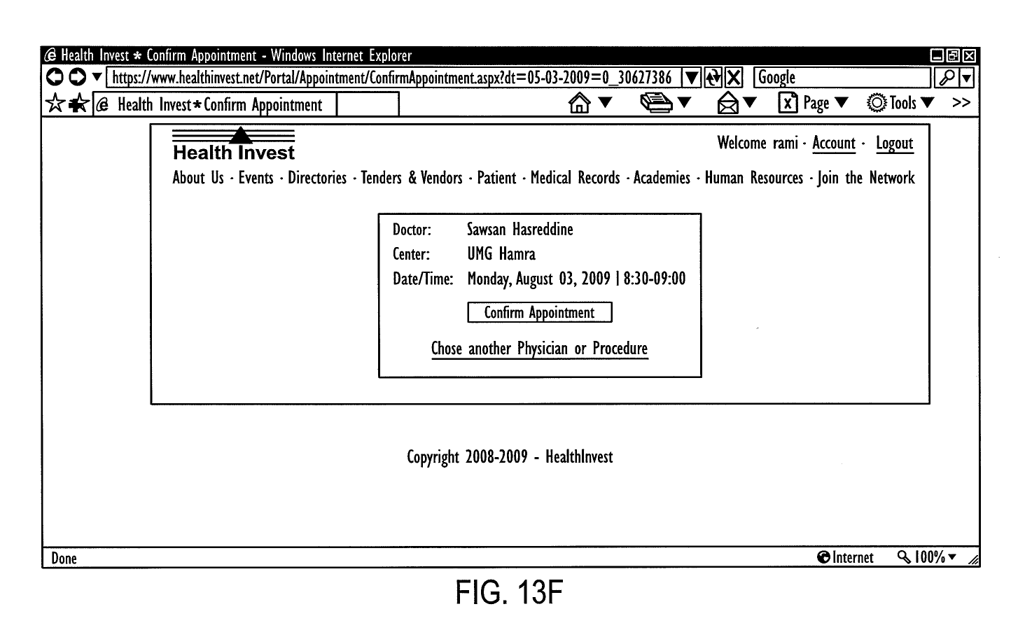

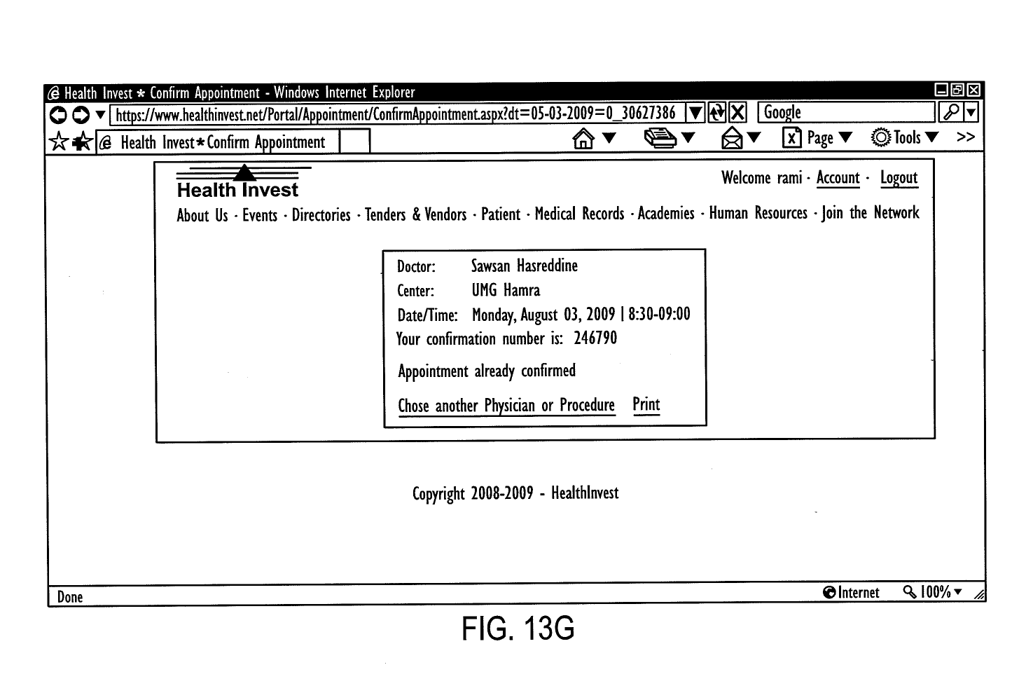

If the user is, for example, a physician (illustrated, e.g., in FIGS. 4A-4R and corresponding screen shots FIGS. 12A-12R) and he/she has been granted access to the system 100, the physician is presented a home screen via the healthcare provider interface 142. The physician is then presented with a plethora of options to choose from. The physician, can, for example, update his/her profile, including contact information, specialty, adding/deleting credentials, etc. via the healthcare provider interface 142. The physician can also search for patients seeking treatments corresponding to their specialty, assuming information provided by a patient is marked for public access (described in more detail herein).

The global health networking system 100 is configured to allow the physician to view a specific patient's electronic medical records. This option requires an added level of security, as illustrated in the screen shot of FIG. 12B where the physician is required to enter a plurality of numerical codes (as previously described) in order to be granted access to confidential medical files. Once the numerical codes are entered, the physician is granted access to the patients home screen including profile, contact information, electronic medical records, etc. See. e.g., FIG. 4B. Once the physician is granted access to a patient's home screen, the physician is permitted to maneuver within the home screen and update specific medical information. For example, the physician can enter his/her specialty in the file history (see, e.g., 4C & corresponding screen shot 12C), record visit (appointment) information, view information, and print visit information (see e.g., 4D-4F and corresponding screen shots 12D-12F). The physician can view, add, delete, edit, and/or complete the medical history of patient and/or family of the patient (see, e.g., FIGS. 4G and 4H and corresponding screen shot 12F and 12G). By selecting, for example, the “History” link from the menu, the physician can select “Edit Family History” or “Edit Medical History” and enter information. By saving and closing the history, the physician and/or patient can view the information at a later time.

In addition to viewing a patients electronic medical records, the physician can enter, view, and print laboratory and radiology (i.e., x-ray) results. As shown in FIGS. 4I-4N and corresponding screen shots 12H-12N, by selecting the laboratory link (“lab” as illustrated in FIGS. 4I-4K), the physician can select “Fill Result” to enter laboratory result information (see, e.g., FIGS. 4I, 12H, and 12I). The physician can select the “Filled Labs” and, subsequently, the “View Results” to view laboratory results and further edit and/or print the information (see, e.g., FIGS. 4J, 4K, 12J, and 12K). Once the system 100 displays the selected laboratory results, the physician can further edit the information and also print the information by selecting the “Print” link. Similarly, as shown in FIGS. 4L-4N and corresponding screen shots 12L-12N, by selecting the Radiology Link, the physician can enter radiology information, view radiology results, and print information/results in much the same manner as described above with respect to viewing, entering, and printing laboratory information.

The system 100 is also configured to allow the physician to complete and view a patient's discharge summary. As shown in FIGS. 4O, 4P, and corresponding screen shots 12O, and 12P, by selecting the “Discharge Summaries” link from the menu, the physician can add, edit, or delete a discharge summary. By selecting the “New Discharge Summary” icon, the physician can enter and save new information regarding discharge information for the patient for later viewing.

In addition to the above-described options, the physician can further utilize the global health networking system 100 to request a consultation and to respond to a consultation request. As shown in FIGS. 4Q, 4R and corresponding screen shots 12Q and 12R), the system 100 is configured to allow the physician to search for another physician within the network. By entering search parameters including location (e.g., country, state, city, etc.) and/or specialty, the application system 150 can then search the plurality of databases 130 according to the entered information by the physician. In this case, since the physician is seeking a consultation with another medical professional, the medical search application 152 would search the healthcare provider database 134 to receive appropriate information and provide the information to the requesting physician. The requesting physician can then choose an appropriate physician, and submit a consultation request. For example, if an obstetrician in Washington D.C. wanted to contact a local physician to inquire about potential concerns regarding an enlarged spleen in a pregnant female, the physician would enter desired parameter information. In the instant case, because an enlarged spleen can be an indication of certain types of blood disorders and/or cancers, which would typically be outside an obstetrician's area of specialty, the obstetrician might enter parameter information including “oncologist” or physician with a specialty of oncology, desired location (e.g., local), and/or the name of the desired medical facility. The medical search application 152 would search the healthcare provider database 134 for oncologists or physicians with a specialty in oncology in the Washington D.C. area. The application system 150, including the medical search application 152, would provide appropriate search results to the physician via the healthcare provider interface 142. Once the physician chooses an appropriate oncologist, the physician can submit a consultation request and include any information pertaining to the information needed.

The consultation request can be submitted in any suitable manner, for example, the physician can choose to submit the request from his/her profile to the requested physician's profile. The request can also be submitted to an email address provided by the requested physician in his/her profile information. The request can also be verbal, via telephone.

The global health networking system 100 is configured to allow the physician receiving the consultation request to respond via the global health networking system (see FIGS. 4R and 12R). Upon logging on to the system 100, the physician in notified of a pending consultation request. The physician can choose to view the requested consultation and can reply to the consultation via the system 100.

### EXAMPLE 2

### Patient

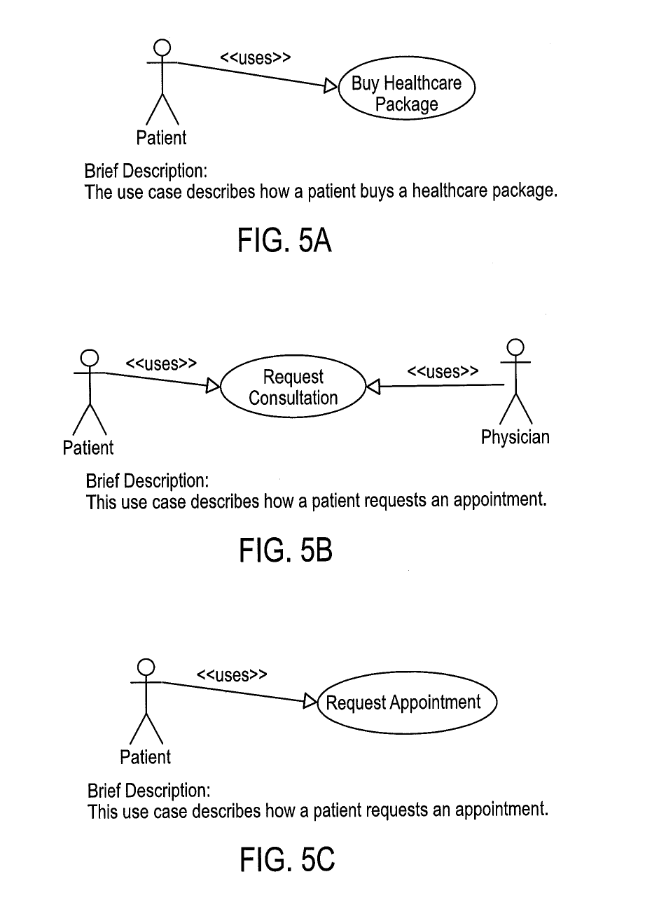

If the user is, for example, a patient (illustrated, e.g., in FIGS. 5A-5C and corresponding screen shots 13A-13G), log in information has been verified, and he/she has been granted access to the system 100, the patient is presented a home screen via the patient interface 141. The patient is then presented with a variety of options to choose from. The patient, can, for example, update his/her profile, including contact information, electronic medical records, desired medical treatment and/or procedure, etc. via the patient interface 141. By default, the system 100 classifies information entered by a patient to be confidential and only visible to the patient upon login after the verification of his/her personal numerical code. The patient, however, can choose to allow certain information to be public and available for viewing by other users (e.g., physicians, other patients, etc.) when the other users search the system 100. This allows other viewers to contact the patient via the system 100 to provide physician recommendations, information regarding personal experiences, support, etc. The patient can also search for other patients with medical conditions similar to their own or for patients seeking treatments similar to treatments/procedures desired by the patient, assuming information provided by other users is marked for public viewing. If not marked for public viewing, the patient would not be able to access confidential information without the other users personal numerical code, as previously described.

In addition to the above noted options, once a patient is logged onto the system 100, the patient can further search for and purchase a healthcare package, as shown, for example, in FIG. 5A and corresponding screen shots 13A. The system 100 is configured to display search criteria (e.g., country, state, city, provider, product name, service, etc.), and the patient can then search for healthcare packages utilizing the provided search criteria. By selecting search parameters, the application system 150 (as shown in FIG. 1) can then search the plurality of databases 130 according to the entered information by the patient. In the instant case, since the patient is seeking information from potential health insurance companies (e.g., corporate subscribers), the medical search application 152 would search the corporate subscribers database 132 to receive appropriate information. The system 100 would then display a list of packages corresponding to the search criteria entered by the patient. The patient can select a desired package for purchase. Upon entry of and validation of payment information by the patient and system 100, respectively, the patient can print a confirmation page for his/her records and can also print a healthcare ID card representing the healthcare package purchased. In the case where the patient does not have a user name, password, and personal numerical code, the system 100 is configured to prompt the patient to register with the system 100 as a new patient and open a new patient account. Once registered, the new patient can search and purchase healthcare packages as previously described.

Similar to consultation requests from one physician to another, a patient can request a physician consultation (e.g., phone, e-mail, live-chat, etc.). As shown in FIGS. 5B and corresponding screen shots 13B and 13C, by entering search parameters including location (e.g., country, state, cite, etc.) and/or specialty, the application system 150 (shown, for example, in FIG. 1) can search the plurality of databases 120 according to the entered information by the patient. Similar to above, with respect to physician-to-physician consultation, since the patient is seeking a consultation with a medical professional, the medical search application 152 would search the healthcare provider database 134 to receive appropriate information and provide the information to the patient. The patient can then choose an appropriate physician (i.e., a physician with a specialty most suited to the patients needs), and attach a document with the consultation message including information pertaining to the reasons the consultation is requested and can further request a desirable method of contact for the consultation (e.g., in person, telephonically, electronically via live-chat, etc.) Unlike the requests made from physician to physician, the patient request requires payment. Once the consultation message is created, the system 100 is configured to prompt the patient to enter payment information, validate the payment information, and confirm the payment information. Upon verification of payment, the system 100 is configured to send the consultation message to the physician.

The patient can further utilize the global health networking system 100 to schedule an appointment or to request a physician consultation. As shown in FIGS. 5C and corresponding screen shots 13D-13G, once the patient is logged onto the system 100, the system 100 is configured to allow the patient to search for a physician within the network 110. By entering search parameters including location (e.g., country, state, cite, etc.) and/or specialty, the application system 150 can then search the plurality of databases 130 according to the entered information by the physician. In this case, since the patient is seeking to schedule an appointment with a medical professional, the medical search application 152 would search the healthcare provider database 134 and/or medical facilities database (not illustrated) to receive appropriate information and provide the information to the patient. The patient can then choose a desired physician and/or medical facility to request an appointment with. Once the physician is chosen, the system displays a calendar indicating dates available for scheduling (see, e.g., screen shot 13C), and allows the patient to select a desired day. They system 100 then displays time slots available for the desired day for the scheduled appointment, allows the patient to select a desired time, and prompts the patient to confirm the selected appointment. Once the appointment is confirmed by the patient, the system 100 confirms the appointment by displaying the appointment details and further provides a confirmation number (see, e.g., screen shot 13E).

In addition to the above, the global health networking system 100 includes at least one subsystem (i.e., medical tour group system) configured to allow the patient to reserve a customized medical tour as well as a customized medical group tour. A customized tour can include a medical procedure, transportation accommodations, overnight lodging, meetings, dining, and/or activities/events, etc.

FIG. 20 is a flow chart 400 illustrating the operation of the global health networking system 100 of FIGS. 1 and 2, wherein the user's needs are linked to information stored in the plurality of databases 130 allowing continuous real time access to medical personnel, medical files, electronic medical records, etc. The flow chart 400 further illustrates a method for reserving a medical tour. In such operations, the user can create a customized medical tour according to information provided such that the medical tour includes a desired medical procedure, a desired medical facility, desired transportation accommodations, and desired lodging.

At step 401, the security module of the server 120 presents the user with a logon/authentication page via a user interface in order to establish secure communication and provide authorized access of the server 120 via the network 110. An example of the logon/authentication page is illustrated, for example, in the screen shot of FIG. 12A. Once the user enters his/her user name, password, and/or personal numerical code, the user information is verified by the system 100. Subsequently, at step 402, the system 100 presents the user with a home screen. The system 100 is configured to control the user's access to capabilities and features within the home screen according to the login information provided. Based on the level of access granted, at step 403, the system 100 presents a plurality of options (at the user's home screen) via the user interface by way of visible graphical interface features (e.g., menus, icons, links, dialog boxes, etc.) such that the use can maneuver through the system 100 and enter data.

At step 404, the system 100 presents the user with a browser page according to the user's selection. In a first example, if the user chooses to search physicians within a desirable mileage radius, the user can access the application system 150. The global health networking system 100 will present the user with a browser page prompting the user to enter search parameters for physicians including, for example, location, specialty, etc. In a second example, if the user chooses to search for desired medical procedure, the same application system 150 is used as described above. The system 100 will present the user with the browser page prompting the user to enter search parameters. In this case, the user would not complete search parameters for a specific physician but rather for a medical procedure. Search parameters can include, for example, type of medical procedure, desired cost, location, etc. In a third example, if the user chooses to search for individuals desiring similar medical procedures, the user can enter search parameters on the browser page for locating patients. Search parameters for searching patients can include, example, gender, type of medical procedure, location, etc.

At step 405, the global health networking system 100 accepts the search parameters entered by the user and at step 406, the application system 150 searches the plurality of databases 130 according to search parameters entered by the user. More specifically, as previously described herein, the application system 150 includes a plurality of search applications 152-158 (e.g., the medical search application 152, the accommodation search application 154, the patient search application 156, the reservation application 158, etc.) In the first example from above, when the user chooses to search physicians, the global health networking system 100 accepts the search parameters entered by the user. The medical search application 152 within the application system 150 then accesses and searches the healthcare provider database 134. In the second example from above, when the user chooses to search for a desired medical procedure, the system 100 accepts the search parameters. The medical search application 152 within the application system 150 the accesses and searches medical procedure database (not illustrated) according to the search parameters entered by the user. In the third example from above, when the user chooses to search for individuals desiring similar medical procedures, the user can select the link for individual search and enter search parameters. The system accepts the search parameters, and then the patient search application 156 is activated to access and search the patient database 136 according to the search parameters entered by the user.

At step 407, the application system 150 receives information from the plurality of databases 130, and at step 408, presents the information to user. In the first example from above, the medical search application 152 within the application system 150 searches the healthcare provider database 134 and presents a list of physicians according to the search parameters provided by the user. In the second example from above, the medical search application 152 within the application system 150 searches the medical procedure database (not illustrated) and presents a list of the medical procedures according to the search parameters entered by the user. In the third example from above, the patient search application 156 within the application system 150 searches the patient database 136 and presents a list of patients, who have marked their information for public access, according to the search parameters entered by the user.

Alternatively, the global health networking system 100 is configured to combine searches and present a plurality of lists allowing the user to reserve a medical tour or a medical group tour. For example, at step 403, as previously described, the system 100 presents a plurality of options (at the user's home screen). At step 404, if the user chooses to search the system 100 for physicians, medical procedures, etc., the system 100 presents the user with a browser page prompting the user to enter search parameters. The user, at this step, is permitted to enter search parameters for physicians (e.g., location, specialty, etc) and for a desired medical procedure (e.g., type of medical procedure, desired cost, location, etc). Additionally, the user is allowed the option of searching for other patients, who have marked information for public access, interested in the similar procedures (described in more detail herein).

At step 405, the system accepts the search parameters entered by the user and at step 406, the application system searches the plurality of databases 120 according to search parameters entered by the user. In the present case, the medical search application 152 within the application system accesses and searches the healthcare provider database and the medical procedure database according to the search parameters entered by the user. The patient search application 156, if opted by the user, accesses and searches the patient database.

At step 407, the application system receives information from the healthcare provider database and the medical procedure database, and at step 408, presents the information, in the form of lists, to the user. More specifically, in the present example, the medical search application presents either a list of physicians who perform the medical procedure requested or a list of the medical procedures offered by physicians according to the search parameters. The system, thus, allows a user the option to choose the desired medical procedure at a desired location, at a desired cost, and/or performed by a desired physician. The system, if opted by the user, present a list of other patients seeking similar medical procedures. The system is configured to filter the list according to age, gender, location of patient, according user preferences entered.

Once the user has chosen the medical procedure at the desired location, cost, etc., the system, at step 409, prompts the user to enter accommodation preferences. Accommodation preferences can include, for example, lodging (e.g., hotel, motel, bed and breakfasts, resorts, etc.) and transportation (e.g., air travel, train, rental car, etc.). The lodging and transportation preferences can also include destination, arrival and departure dates, number of members in the group, and group-type information, etc. If the user chooses to enter accommodation preferences, the system 100, at 410, prompts the accommodation search application 154 to access and search the corporate subscriber database 132, which stores accommodation information provided by corporate subscribers.

At step 411, the application system 150 places queries to the network of corporate subscribers in the corporate subscriber database 132, including lodging and transportation information. At step 412, the system 100 presents the user with information based on the search parameters provided by the user. More specifically, in the present example, the accommodation search application 154 presents lists pertaining to lodging and transportation rates and availabilities, according to the user's preferences. For example, the lists can include availabilities for airlines, airplane seats, lodging rooms, bus seats, train seats, etc., and their associated costs. Thus, in addition to allowing a user the option to choose the desired medical procedure at a desired location, at a desired cost, and/or performed by a desired physician, the system also provides the user information for reserving transportation to and from the desired location, and lodging accommodations at or near the desired location. As such, the system allows the user to create a fully detailed customized medical tour including a desired procedure within a desired price range and at a desired location.

Once the desired medical procedure and accommodations are selected, the system 100, at step 413 prompts the user to reserve the customized medical tour. More specifically, the reservation application 158 of the application system 150, at step 413, prompts the user to submit a reservation request to the medical tour group system for the selected medical procedure, lodging, and/or transportation accommodations. Upon requesting the medical tour reservation, the reservation application 158 reserves the medical procedure with the medical facility associated with the chosen procedure and reserves the lodging and transportation accommodations and proceeds to payment information.

At step 414, the system prompt the user for payment information. The user if permitted to enter any form of suitable payment (e.g., credit card, debit card, pay pal, direct billing, etc), and can also provide health insurance information if the desired procedure is provided coverage. Alternatively, the user can choose to provide an initial deposit and pay the remainder in full upon completion of the procedure, upon checkout of their lodging choice, etc.

Finally, at step 415, the system provides a confirmation of the medical tour including a complete itinerary detailing the individual medical tour participants, the medical tour schedule including, for example, date and time of medical procedure, assigned physician, name and location of hospital, etc. The itinerary can also include the medical tour lodging information including, for example, name and location of lodging, duration of stay, number of rooms reserved, number of guests, cost, etc. and transportation information including, for example, type of transportation, number of travelers, dates and times of departure and arrival, etc., and medical tour events (e.g., area attractions, meals, etc.).

### EXAMPLE 3

### Broker

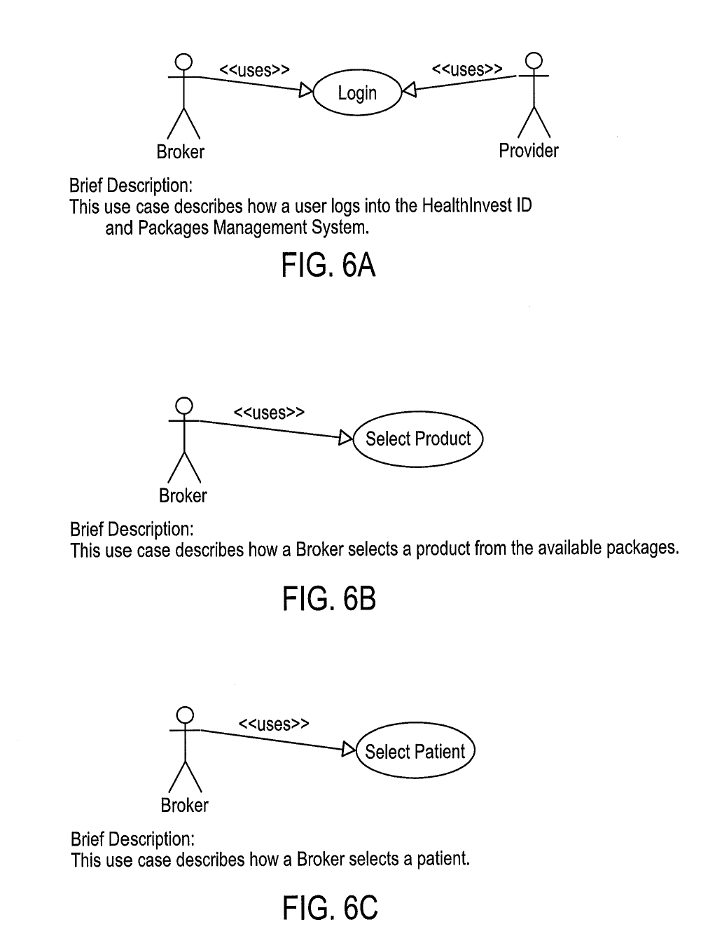

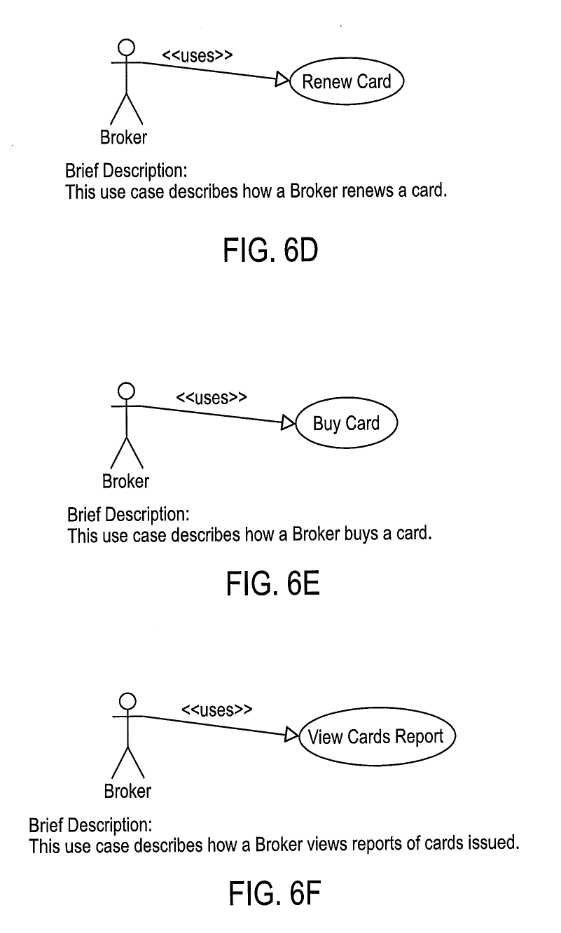

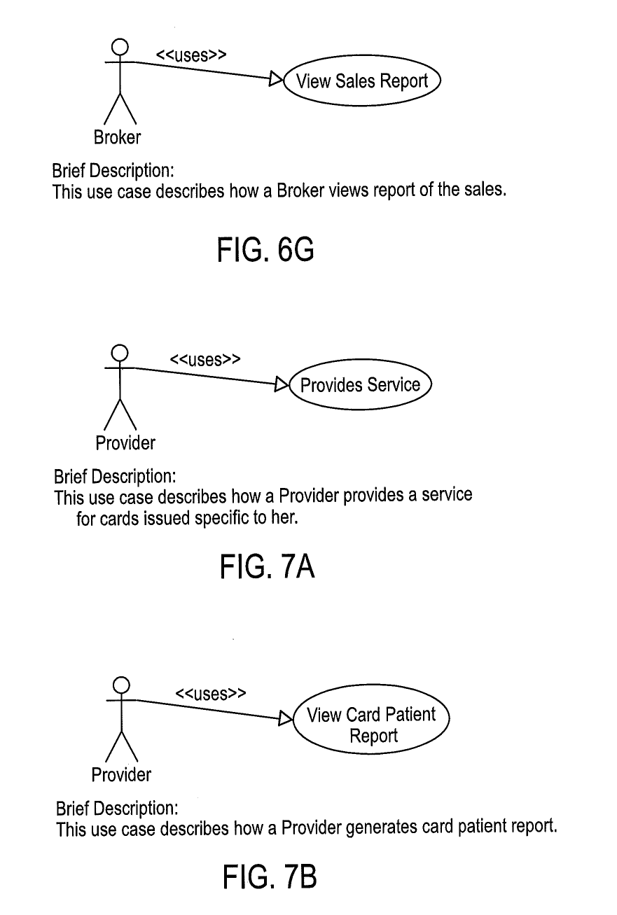

If the user is, for example, a broker (illustrated, e.g., in FIGS. 6A-6G), he/she is prompted to login to the global health networking system 100. Once the login information has been verified, and he/she has been granted access to the home screen, the broker has a variety of options to choose from.

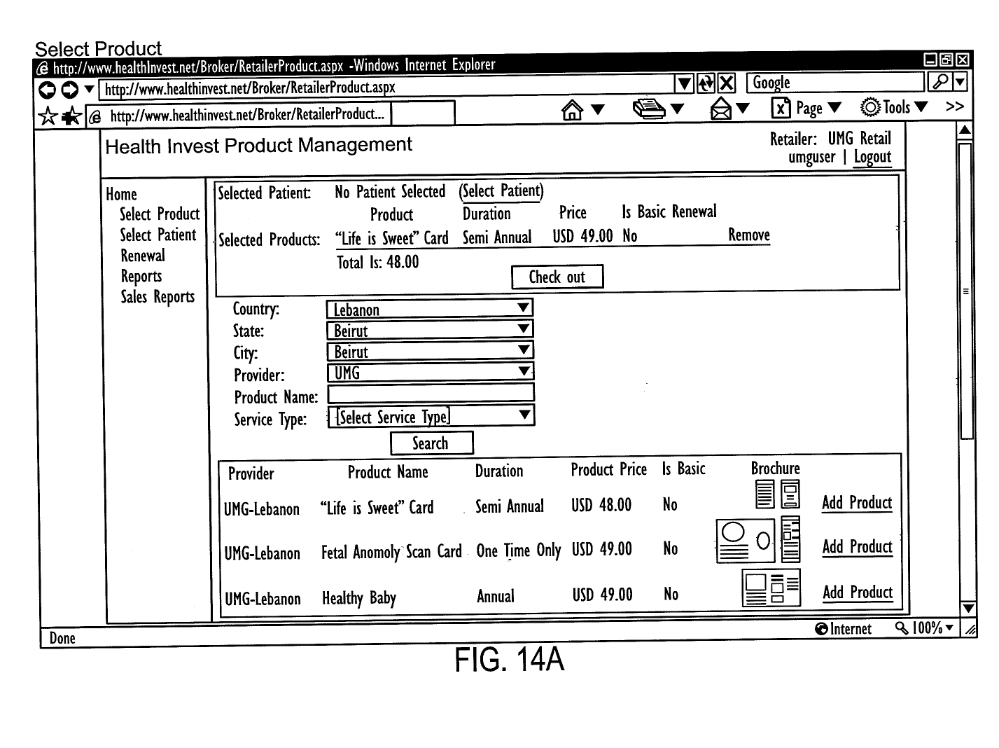

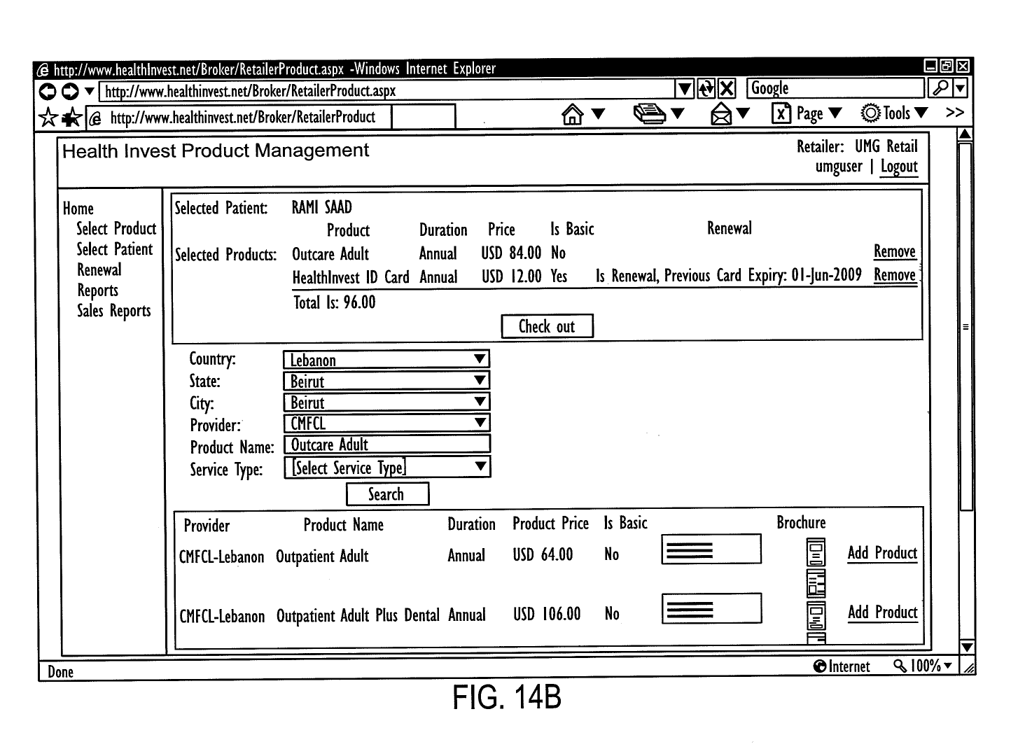

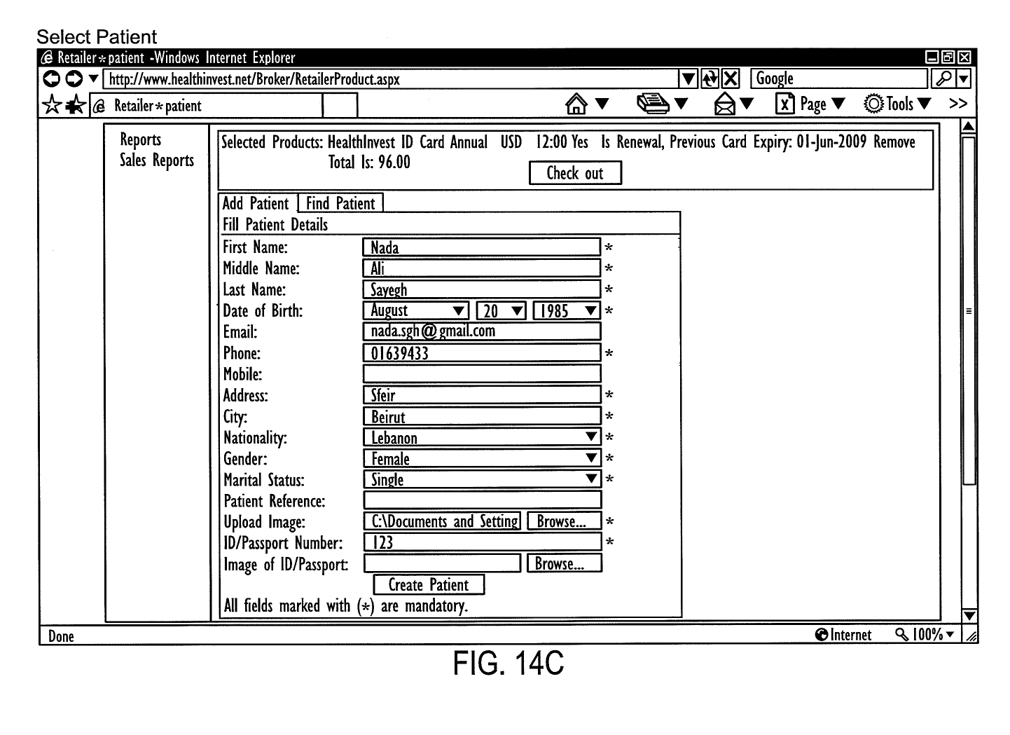

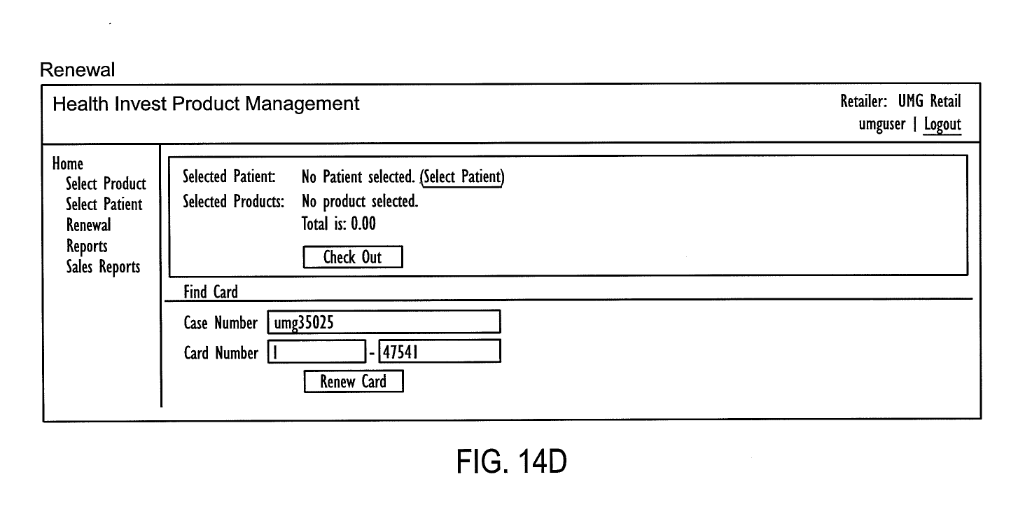

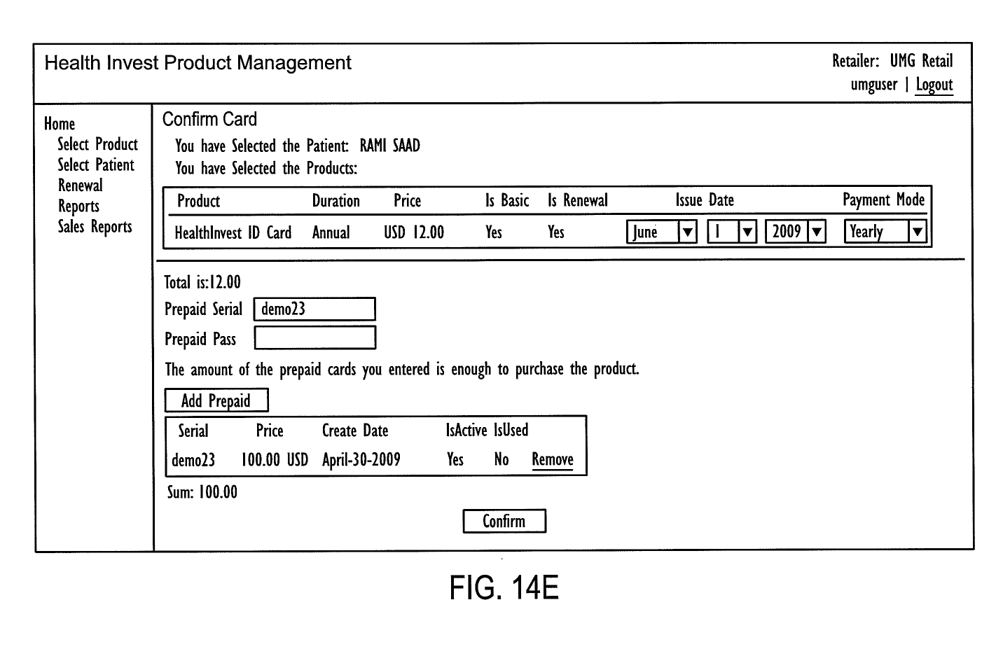

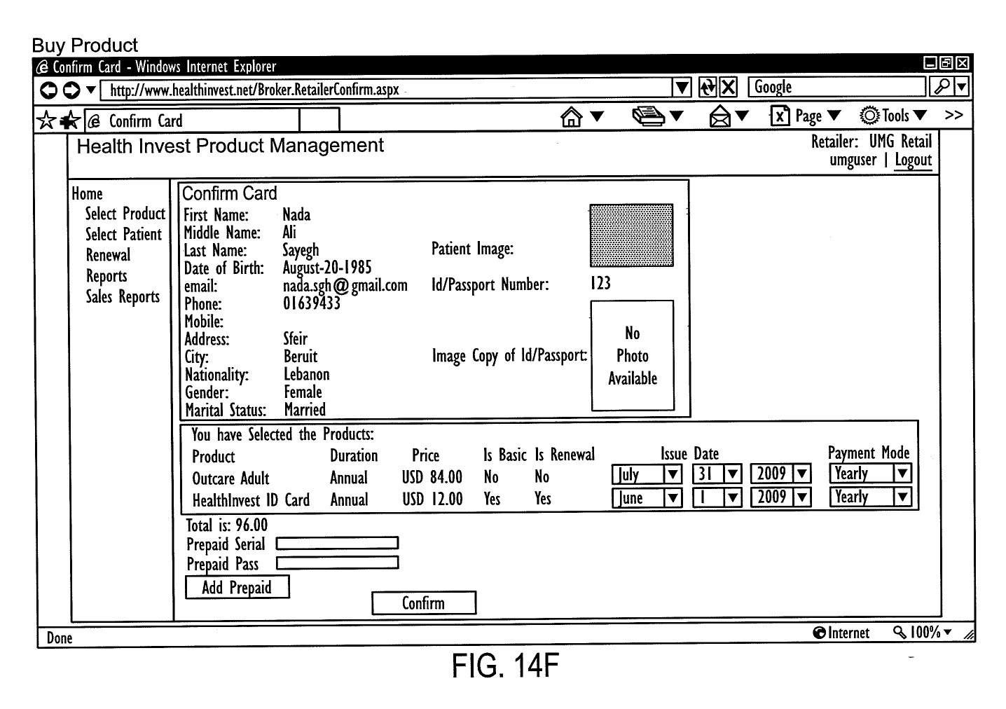

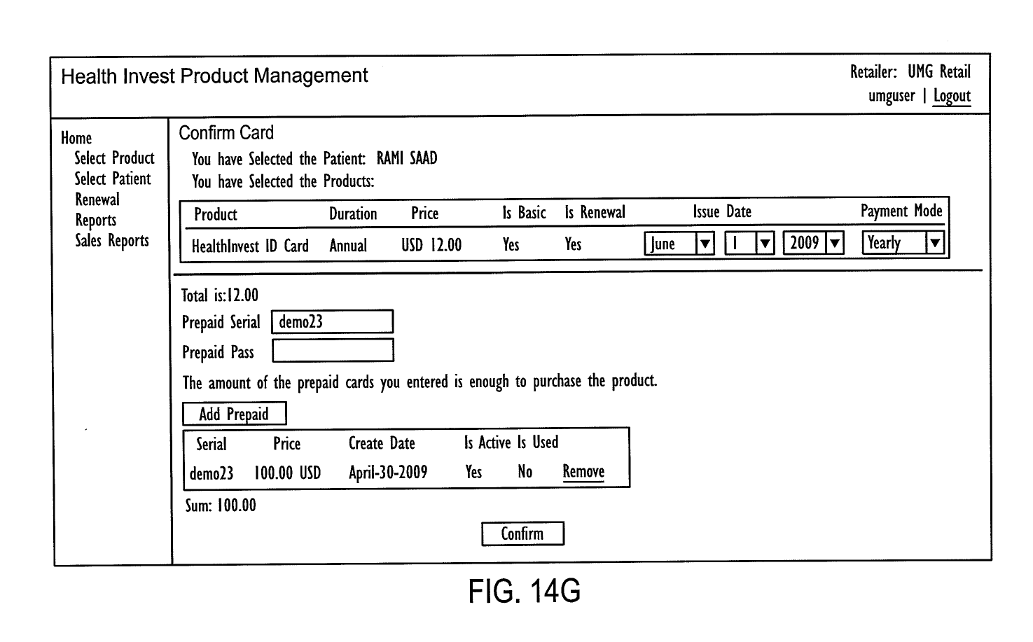

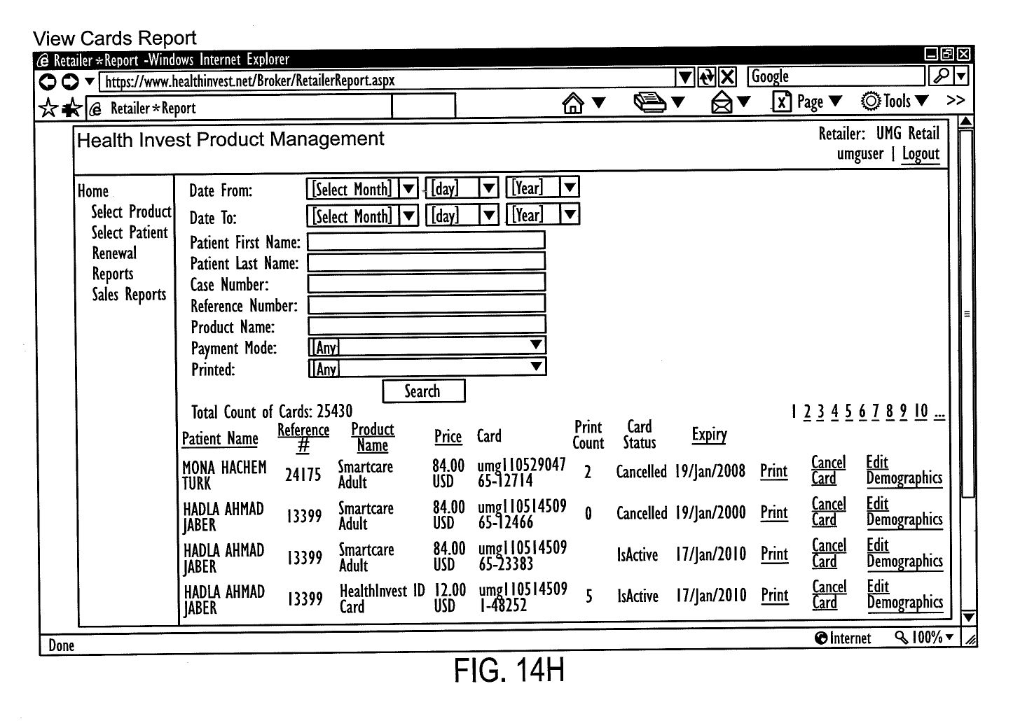

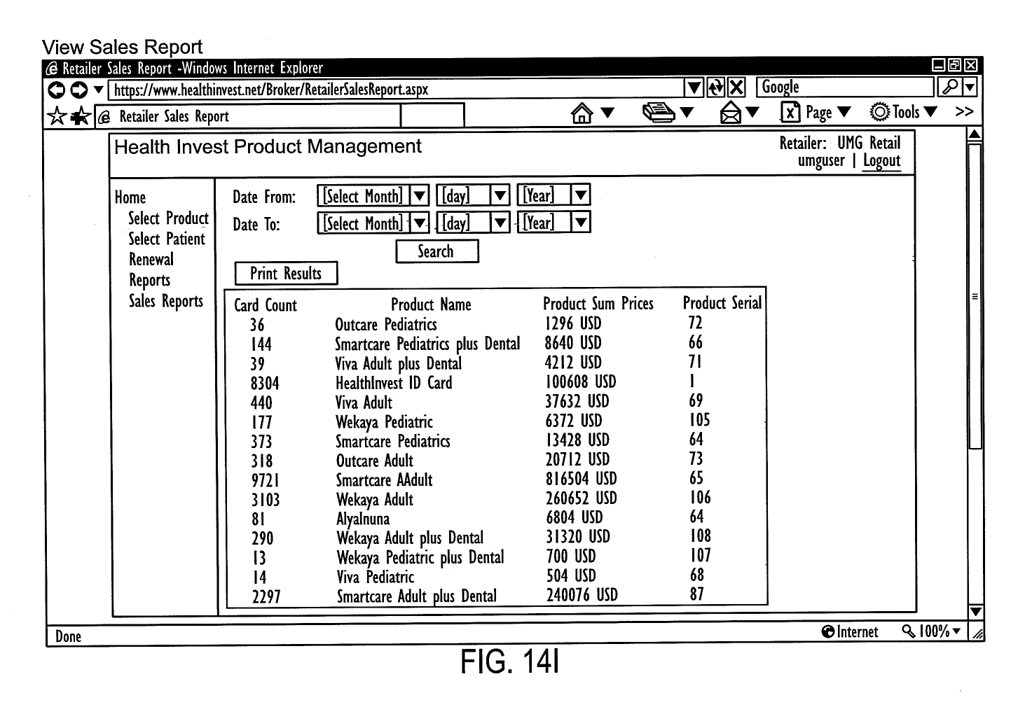

The broker can select products available to authorized users from the packages available on the network 110. For example, as illustrated in FIG. 6B and corresponding screen shots 14A and 14B, the system 100 is configured to allow the broker to search for a product using various search criteria (e.g., country, state, city, provider, product name, service type, etc.). The system 100 then displays a list of products according to the search criteria entered by the broker. The broker can peruse the list of available products and can select a desired product by clicking the “add product” option adjacent to the product in the product list (shown, for example, in FIG. 14B).

The system 100 is further configured to allow a broker to search for patients, as shown, for example in FIG. 6C and corresponding screen shot 14C. The system 100 displays two tabs: “Add Patient” and “Find Patient.” If the “Add Patient” tab is selected, the system 100 prompts the broker to complete patient details and proceeds to display the fields to be completed. Fields marked with an asterisks (*) are mandatory, of which the broker must complete. Once the desired and mandatory fields are completed, the broker can create a patient listing by selecting the “Create Patient” option (see, e.g., FIG. 14C). If the “Find Patient” tab is selected, the system 100 prompts the broker to enter a “Case Number” and a “Card Number.” Once the broker enters both the case number and card number assigned to the patient, the system 100 compares the entered information with the information stored in the databases to locate the patient corresponding to the entered case number and card number.

The system 100 is also configured to allow a broker to renew a patient's ID card and to also purchase an ID card. See, e.g., FIGS. 6D, 6E, and corresponding screen shots FIGS. 14D-14G. The system 100 prompts the broker to enter card information (i.e., card number and case number). See, for example, FIG. 14D. Once the information has been entered, the system 100 compares the entered information with the information stored in the plurality of databases 130 to if the card information is valid. If the card information is valid, the system 100 is configured to display the patient and product information associated with the card. The system 100 then prompts the broker for payment (see, e.g., FIG. 14E) and validates the payment information provided. Payment options can include, for example, prepaid, paypal, pay by mail, VISA, MASTERCARD, DISCOVER, etc. The system 100 finally displays the card renewal information and further provides a renewed ID card for the broker to print. In addition to renewing an ID card, the broker can purchase an ID card (see FIG. 6E), as mentioned above. In this case, the broker enters product and patient information. The system 100 determines if an ID card has already been assigned to the patient. If no record of the patient is found in the databases, the system 100 will then prompt the broker to purchase an ID card, where the broker can choose from a plurality of payment options. Like above, payment options can include, for example, prepaid, PAYPAL, pay by mail, VISA, MASTERCARD, DISCOVER, etc. The system 100 then displays the purchased ID card and further prompts the broker to print.

In addition to the above options, a broker can also utilize the global health networking system 100 to view reports of ID cards issued to patients and also view sales reports. If a broker desires to view reports of cards issued (see, e.g., FIG. 14H), the system 100 displays search criteria for the broker to chose from (e.g., date from, date to, patient's first name, patient's last name, case number, reference number, product name payment mode, printed, etc.). Once the broker enters the desired search criteria, the system 100 searches the plurality of databases 130 for information and displays a grid of information corresponding to the entered criteria. The system 100 is configured to highlight the rows indicating the patients who have a basic ID card. The system 100 also allows the broker to sort the grid by clicking on column headers (e.g., “Patient Name”, “Reference #”, “Product Name”, “Price”, etc.). Furthermore, the broker can print an ID card corresponding to a particular patient as well as cancelling certain ID cards and editing the demographics of the patient in the selected row.

If the broker desires to view reports of ID card sales (See, e.g., FIGS. 6G and 14I), the broker completes the “Date From” and “Date to” fields in the search criteria of FIG. 14H. The system 100 searches the plurality of databases 130 for ID cards issued within the selected date range for that specific broker, and displays a list of products purchased including the card count and the sum price of the cards (see, e.g., FIG. 14I). The broker can then print the results.

### EXAMPLE 4

### Provider

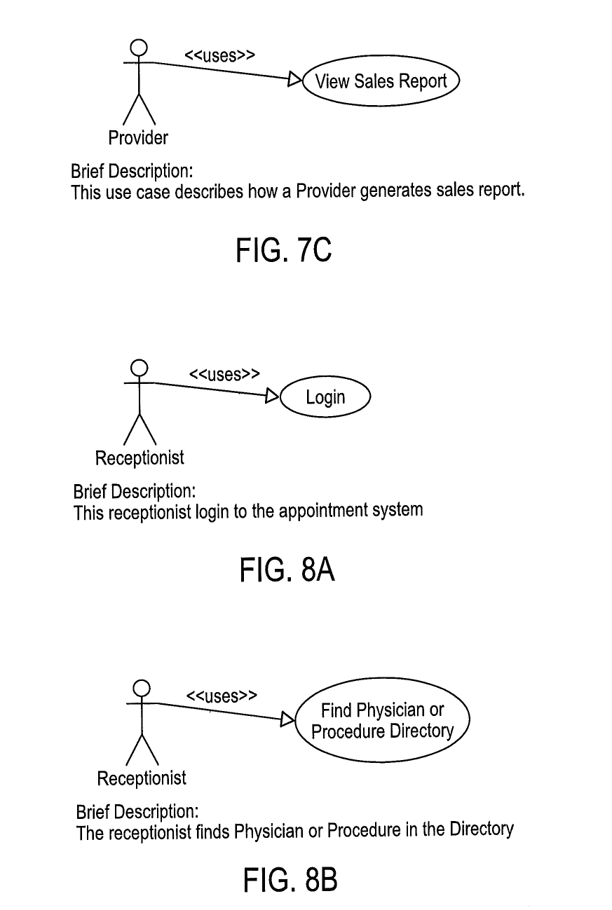

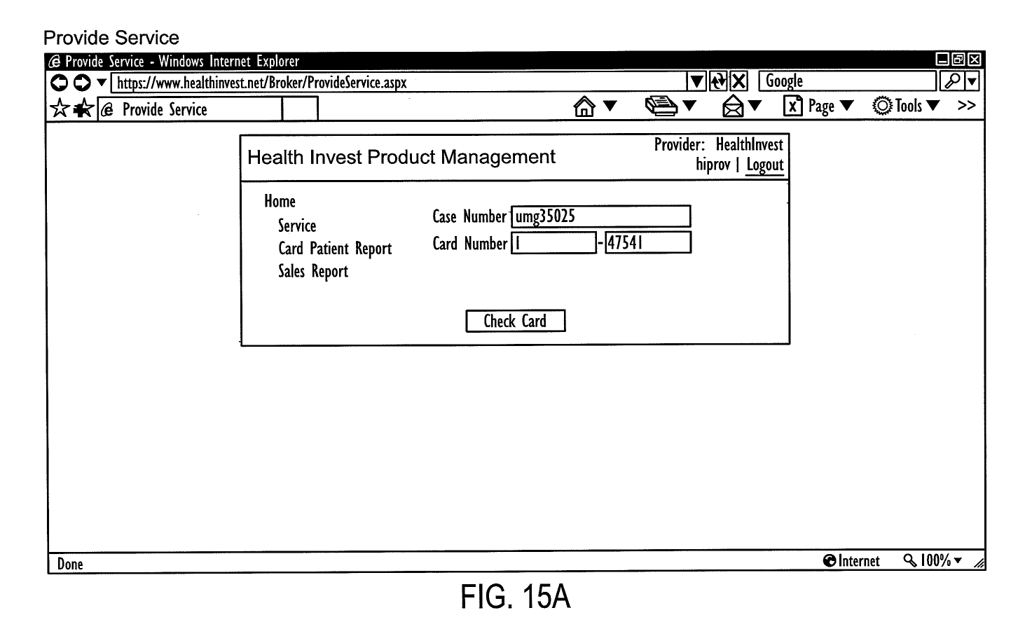

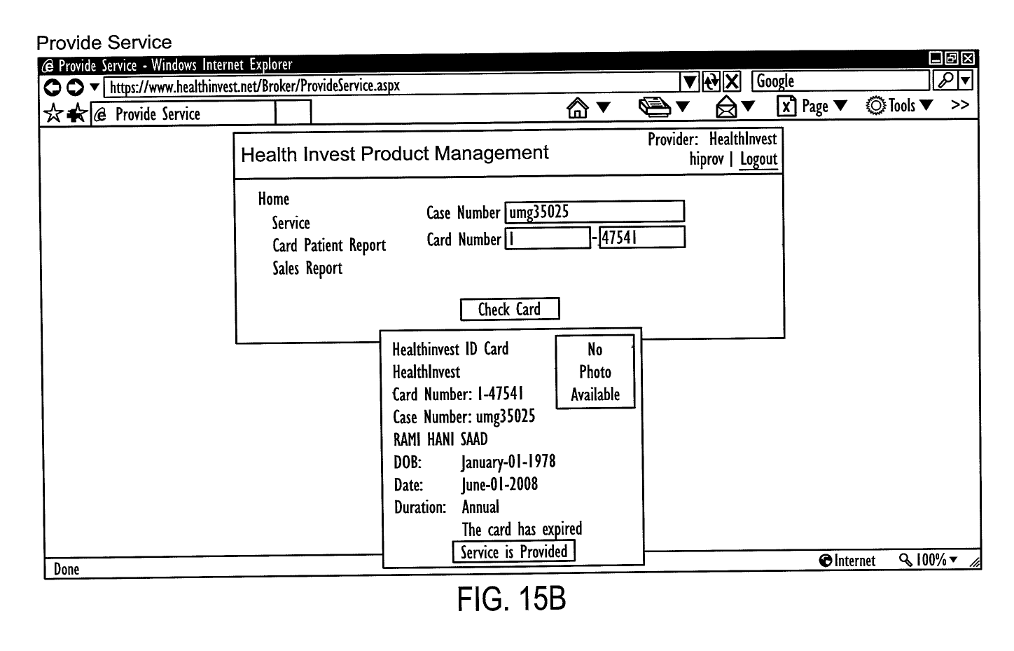

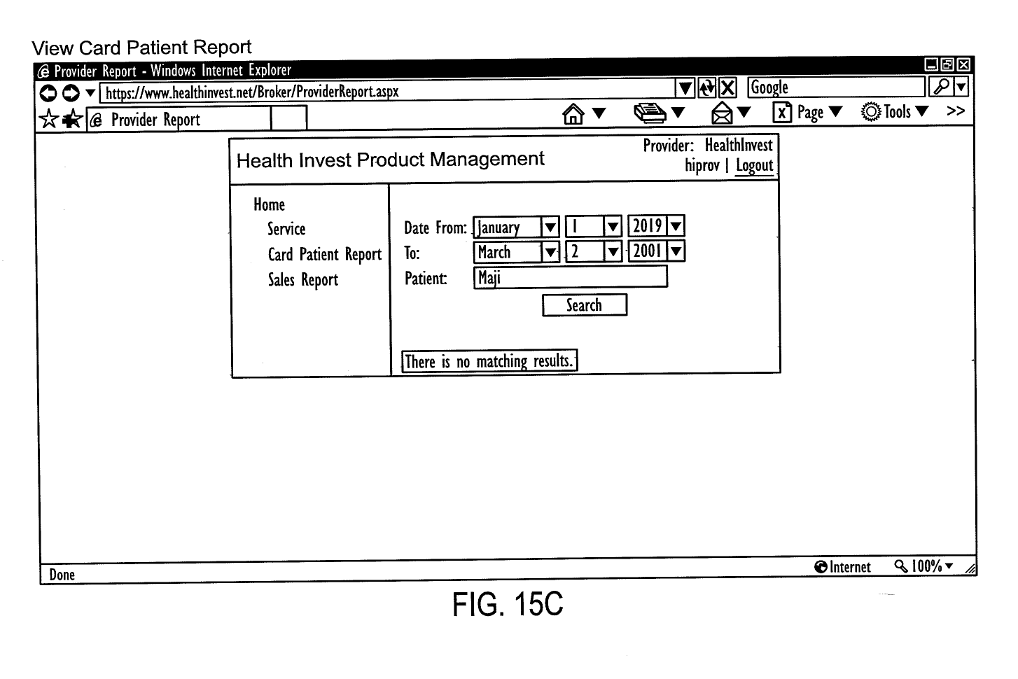

Similar to a broker, as previously described, a provider can access ID card information (see, e.g., in FIGS. 6A-7C and corresponding screen shots 15A-15C). The global health networking system 200 is configured to prompt the provider to enter ID card information (i.e., “Case Number” and “Card Number”). See FIG. 15A. Once the provider enters both the case number and card number assigned to the patient, the system 100 compares the entered information with the information stored in the plurality of databases 130 to locate the patient corresponding to the entered numbers. If the ID card information is valid, the system 100 is configured to display the ID card along with the card status (see FIG. 15B), and further prompts the provider to provide a service for the selected card if the service had not already been provided. Once the service has been entered, the system 100 disables the “provide service feature” and changes the card status to “Active” and “Service is Provided”.

If the provider desires to view a card patient report (see, e.g., FIGS. 7B and 15C), the provider completes the “Date From” and “Date to” fields in the search criteria. The system 100 searches the plurality of databases 130 for ID cards issued to patients within the selected date range for the specific provider, and displays a list of cards and associated patients. Similarly, if the provider desires to view reports of ID card sales (see, e.g., FIGS. 7C and 14I), the provider completes the “Date From” and “Date to” in the search criteria of FIG. 15C. The system 100 searches the plurality of databases 130 for ID cards issued within the selected date range for that specific provider, and displays a list of products purchased including the card count and the sum price of the cards. The provider can then print the results.

### EXAMPLE 4

### Receptionist

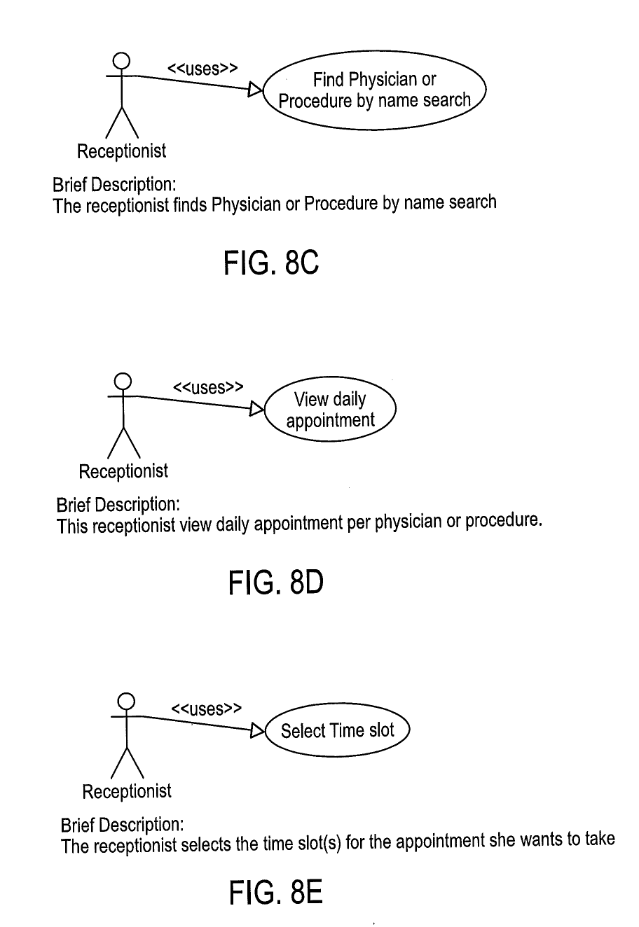

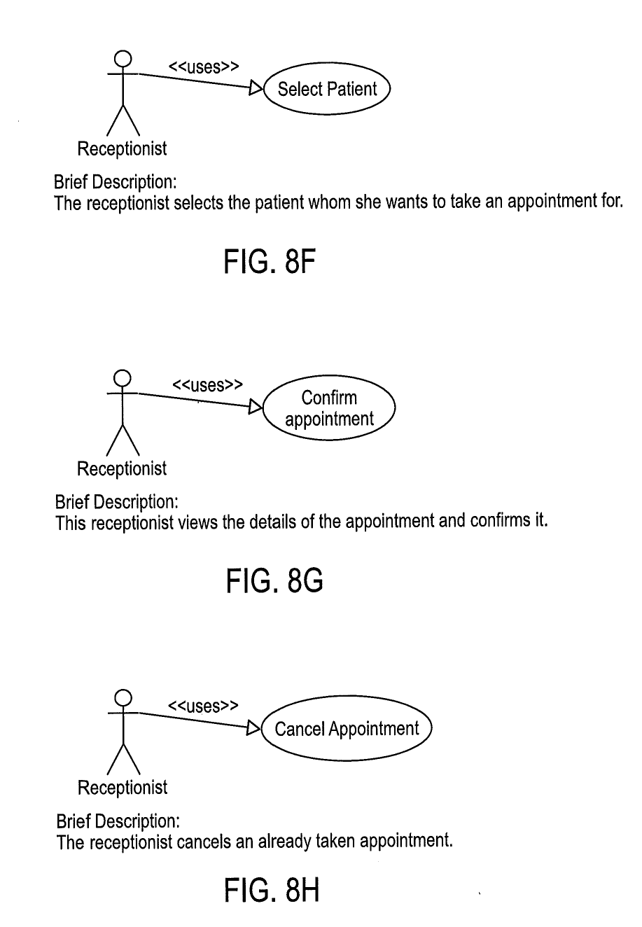

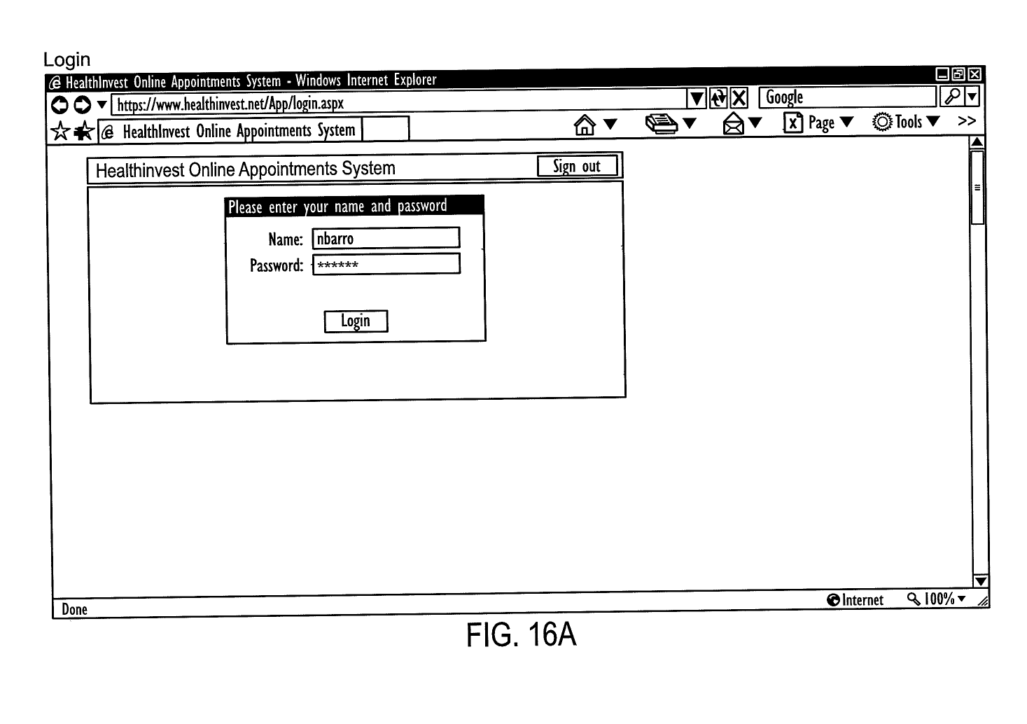

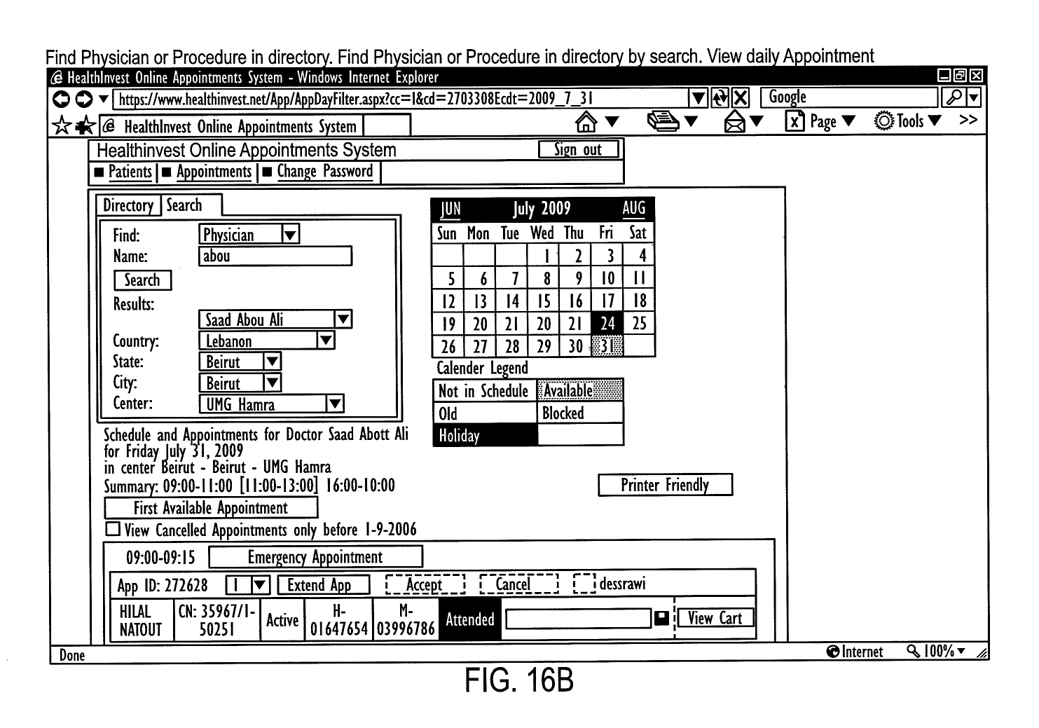

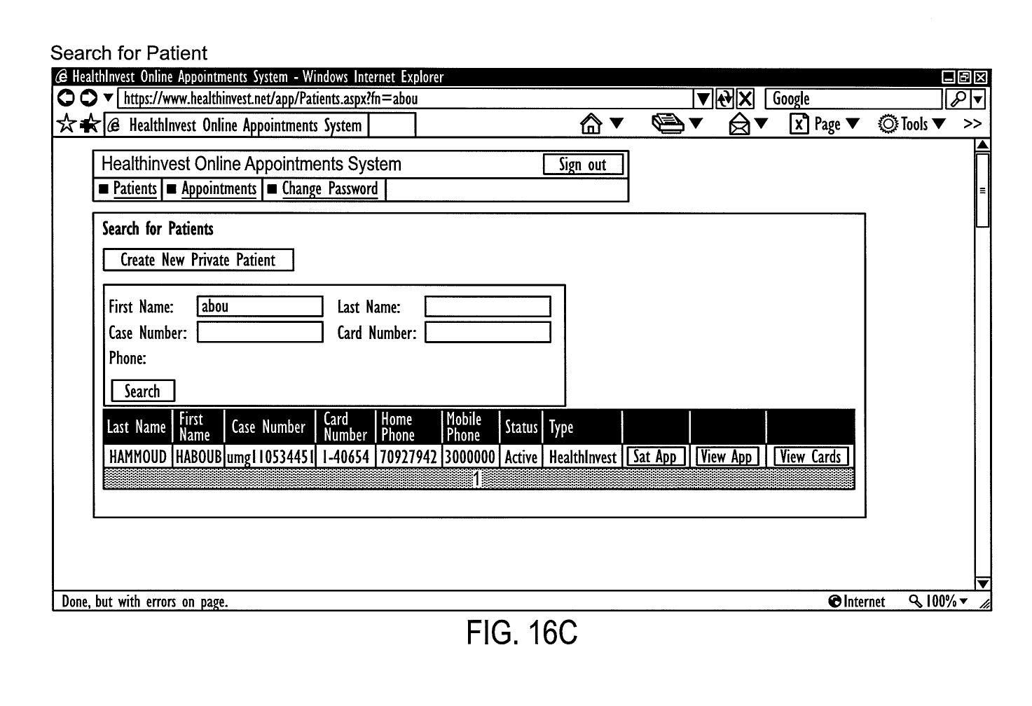

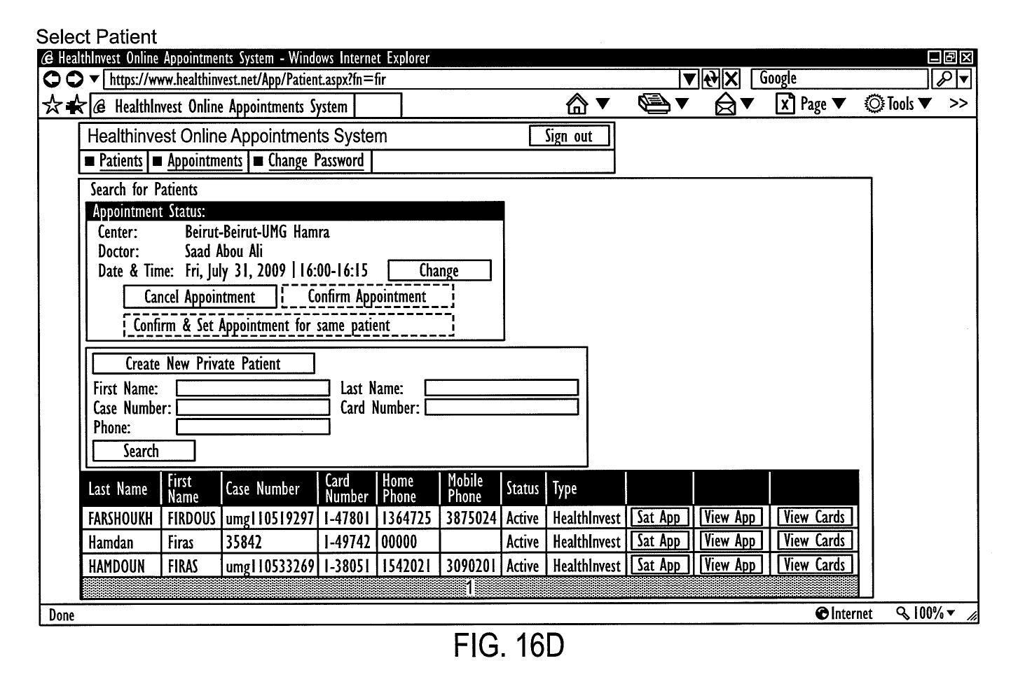

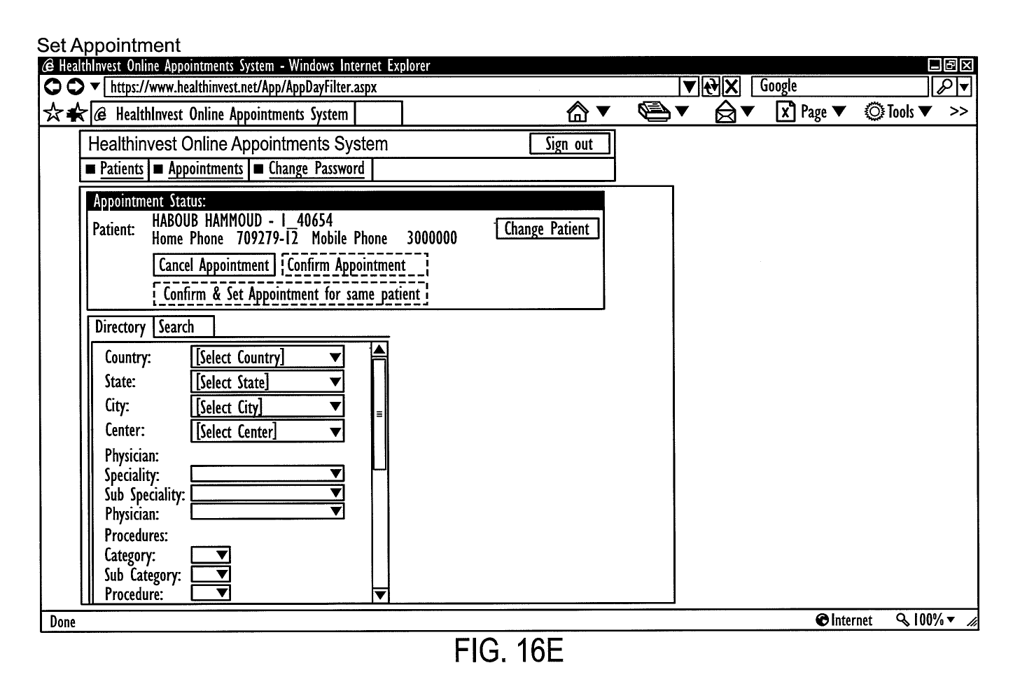

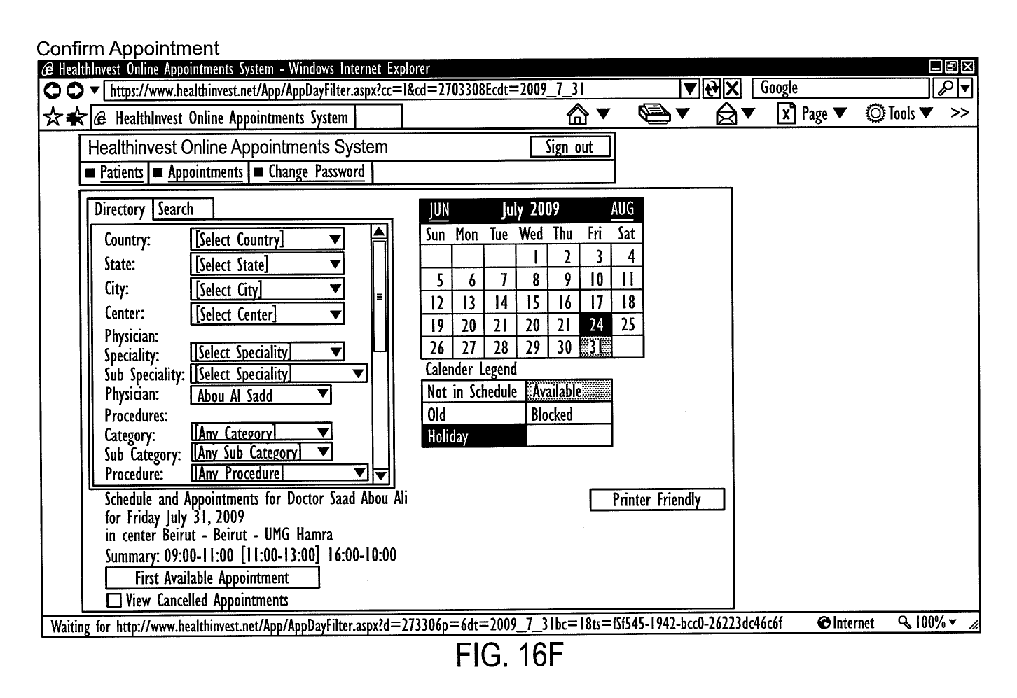

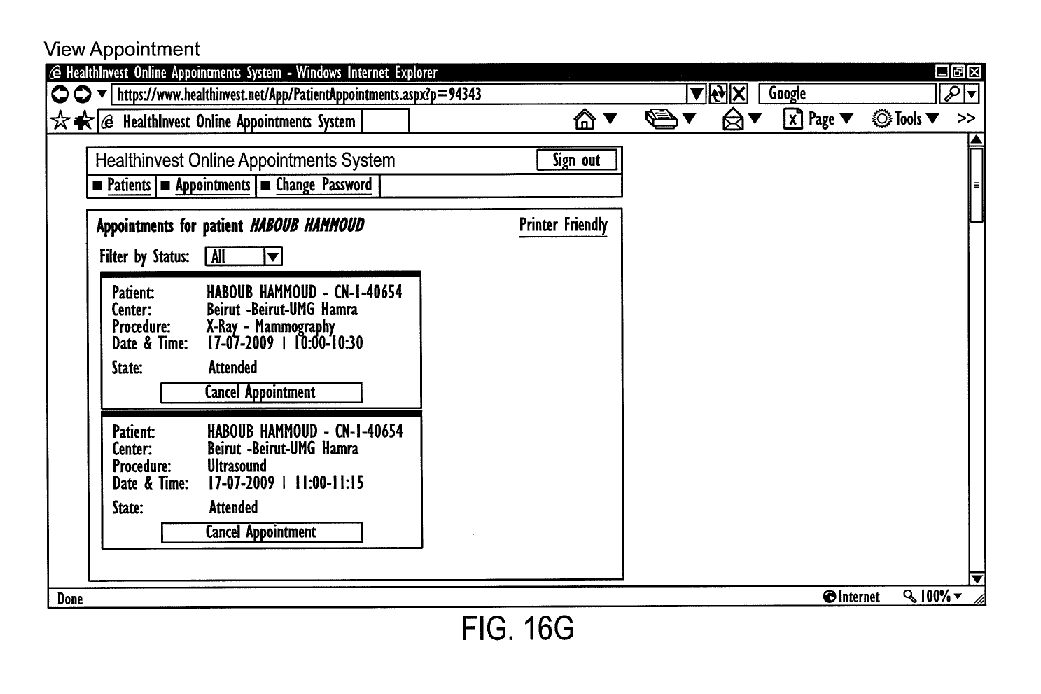

In an aspect of exemplary embodiments of the disclosed system, global receptionists can access the health networking system 100 to assist users in a variety of ways, as shown in FIGS. 8A-8I and corresponding screen shots FIGS. 16A-16F. For example, a receptionist can assist a patient with scheduling an appointment with a physician. In such a case, the system 100 prompts the receptionist to login. The receptionist would then enter his/her username and password, as shown in FIG. 16A. Upon verification of login information, the system 100 displays the global health network's appointments home screen, shown, e.g., in FIG. 16B. Once the receptionist selects the “Appointments” option, the system displays a tab box including a “Directory” tab and a “Search tab” (see, e.g., FIG. 8B). If the receptionist selects the “Directory” tab, search criteria is displayed including, but not limited to, country, state, city, center, physician specialty, physician sub specialty, physician, procedure category, procedure sub category, procedure, etc.). The receptionist would then enter search criteria according to a patient's needs. The receptionist would select the desired country, which then prompts the system to generate a list of states associated with that particular country. The receptionist selects a state, which prompts the system 100 to generates a list of cities associated with that particular state.

If the receptionist desires to locate a physician, he/she selects the patient's desired specialty, which prompts the system 100 to filter subspecialties according to specialty. The receptionist then chooses the desired subspecialty, which prompts the system 100 to generate a list of physicians corresponding to selected subspecialty. The receptionist can then select a desired physician.

If the receptionist desires to locate information regarding availability of a particular procedure, he/she selects a category which prompts the system 100 to filter subcategories according to the selected category. The receptionist then selects the desired subcategory, which prompts the system 100 to generate a list of available procedures. The receptionist can then select a desired procedure.

If the name of the physician or procedure is known, the receptionist can search the system 100 via the known name (see, e.g., FIG. 8C). Similar to above, once the receptionist selects the “Appointments” option, the system 100 displays a tab box including a “Directory” tab and a “Search tab”. If searching by name, the receptionist would select the “Search” tab. The system 100 would then prompt the receptionist to select either “physician” or “procedure”. The receptionist would proceed by completing search box criteria (shown, e.g., in FIG. 16B). Upon selecting the “search” option, the system 100 searches the plurality of database 130 according to the entered search information displays the search results organized by country, state, city, and medical facility (e.g., health center, clinic, hospital, etc.).

In additional to the above patient related assistance, the system 100 is also configured to allow a receptionist to view daily appointments for physicians or for procedures. Once the receptionist enters the physician's name or the name of the procedure, the system 100 is configured to display the monthly calendar corresponding to the desired physician or procedure. See, e.g., FIG. 16B. The calendar can include a legend, and can be, for example, color coded to indicate daily appointment availability. Upon selecting an available day, the system 100 is configured to display the time slots of the selected day, the appointments already scheduled, and time availability for additional appointments. The system 100 also displays information regarding the patient for whom the receptionist is searching, such that the receptionist can view the ID card and previously purchased package information. If the patient desires to schedule an appointment at an available time slot (see, e.g., FIG. 8E), the receptionist selects that particular time slot and selects the “Set Appointment” option. If the patient requires an immediate appointment, the system 100 is configured to allow the receptionist to schedule an “Emergency Appointment” at a time slot already reserved. If the patient requires an extended appointment, the receptionist can choose more than one time slot and selects the “Extend Appointment” option.

The global health networking system 100 is also configured to allow the receptionist to schedule an appointment through entering the patient's information by displaying the patient search box including search criteria fields (e.g., “first name”, “last name”, “case number”, “card number”, “phone number”, etc.). See, e.g., FIGS. 8F and 16C The receptionist completes one or more of the search criteria for the patient and submits a search to the plurality of databases 130. The system 100 then displays a list of patients corresponding to the entered criteria indicating their member status (e.g., active, not active, etc.) as shown, for example, in FIG. 16D. Once a patient is selected, the receptionist can select the “View Appointments” icon to view current appointments for the selected patient and can also select “Set Appointment” to select a patient for whom the receptionist would like to schedule an appointment (see, e.g., FIG. 16E). Once the appointment is scheduled, the system 100 is configured to display details of the appointment (e.g., physician, procedure, patient, date, time, etc.). The receptionist can then select the “confirm” option, which prompts the system 100 to confirm the appointment by displaying the daily time slots with the scheduled appointment shown.

The system 100 is configured to easily allow the receptionist to also cancel a patient's appointment. Once the system 100 displays the daily view of scheduled appointments, the receptionist can select a scheduled appointment and select the “cancel” option, as shown, for example in FIG. 16G. The system 100 then indicates the appointment as “canceled”.

In additional to above noted options available, the receptionist or system administrator can view reports of scheduled appointments. See, e.g., FIG. 8I. The system 100 is configured to display a reports page prompting the receptionist/administrator can enter report search criteria. The search criteria can include, e.g., country, state, city, center, physician, procedure, start date, end date, etc. The system 100 then generates a report organized by the search criteria (e.g., country, state, city, center, physician, procedure, etc.) and displays the number of scheduled appointments, the number of canceled appointments, etc.

### EXAMPLE 5

### Online User

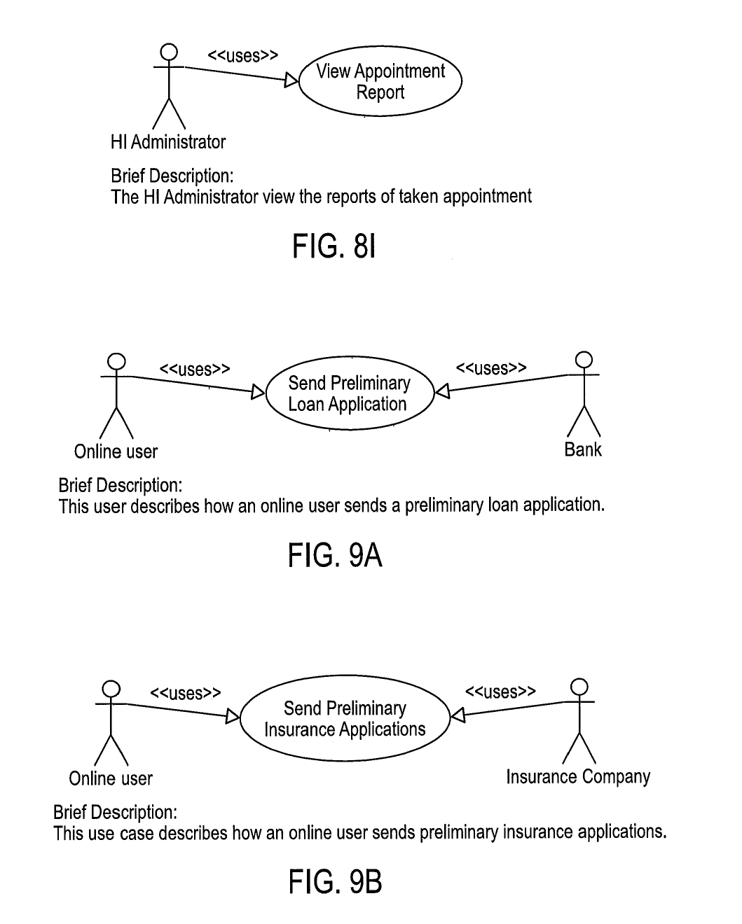

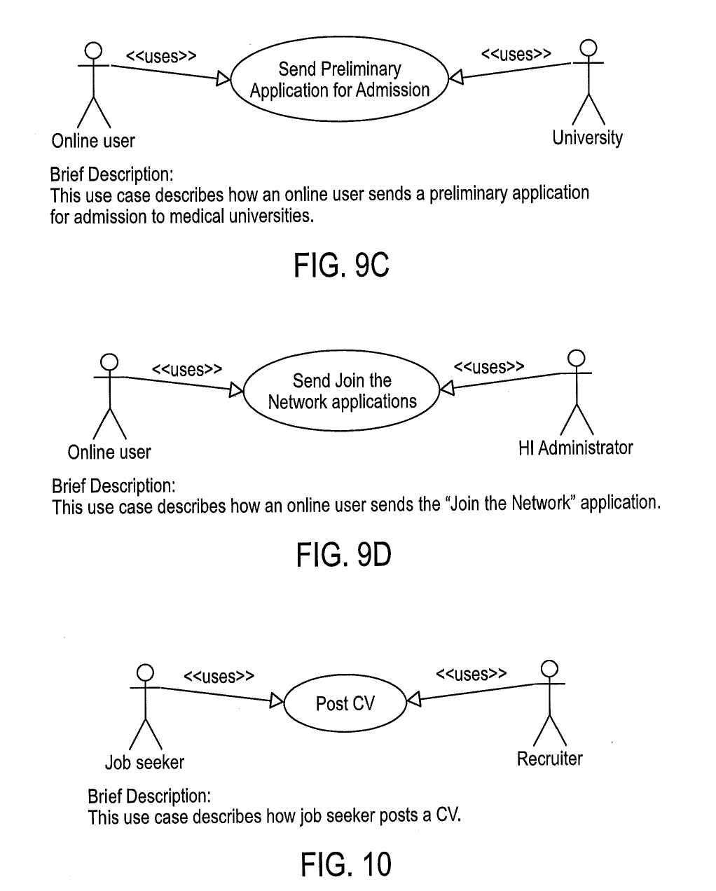

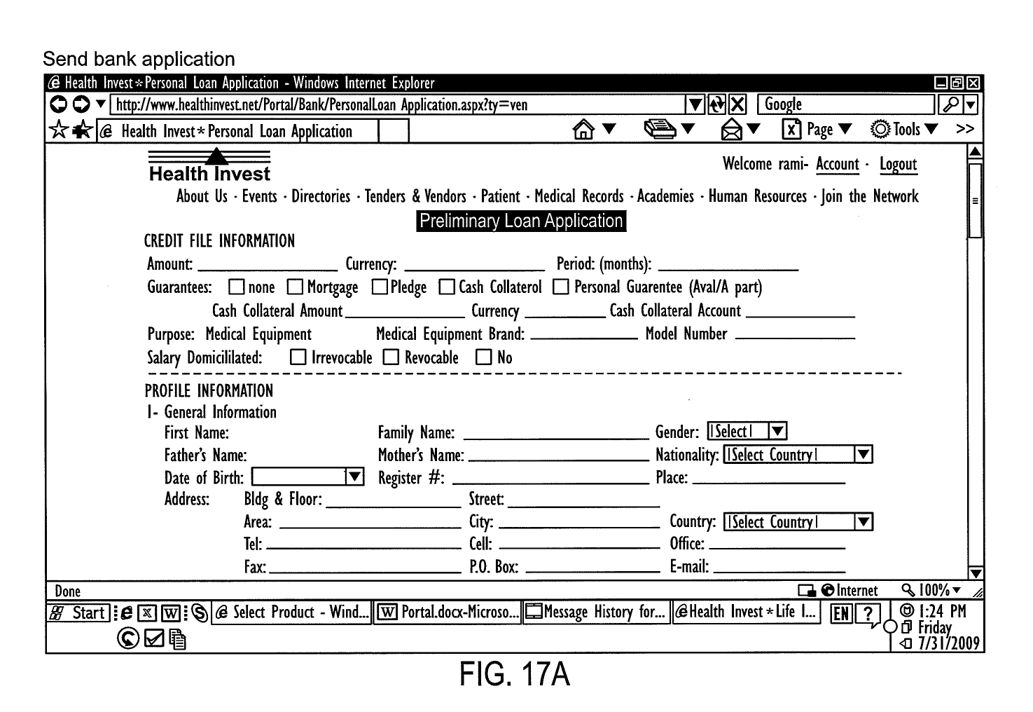

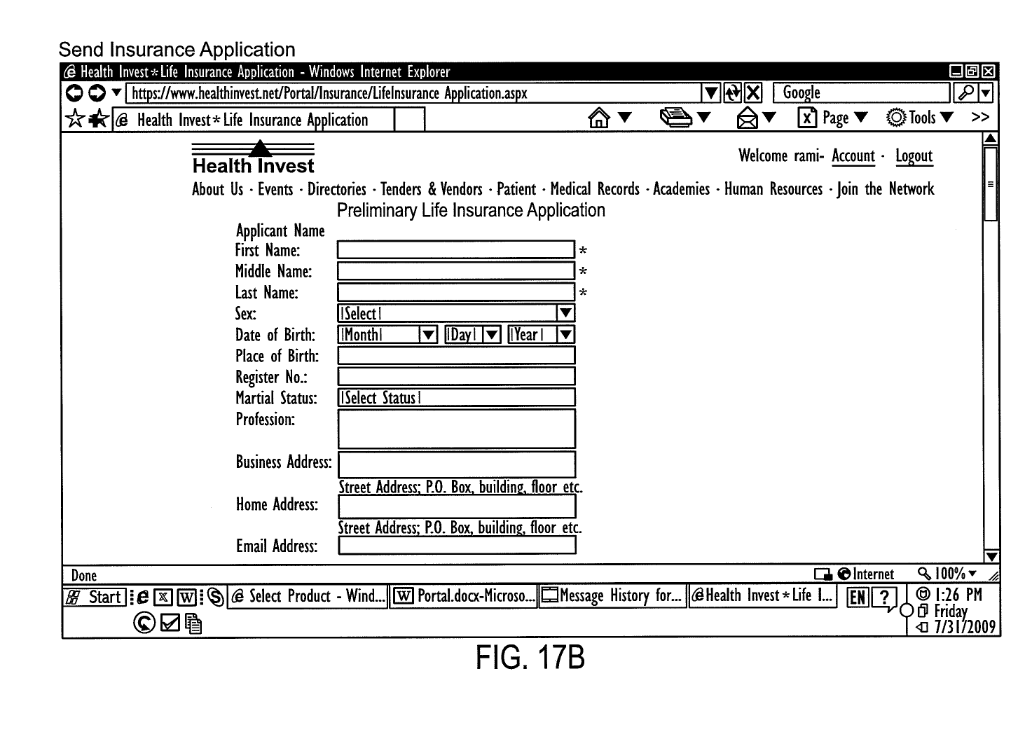

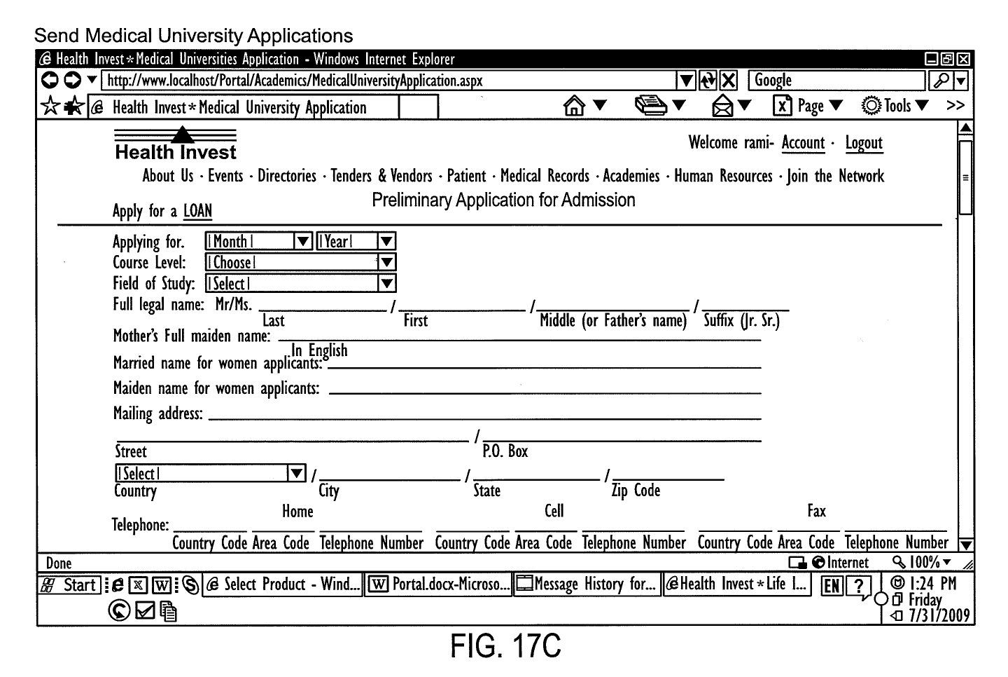

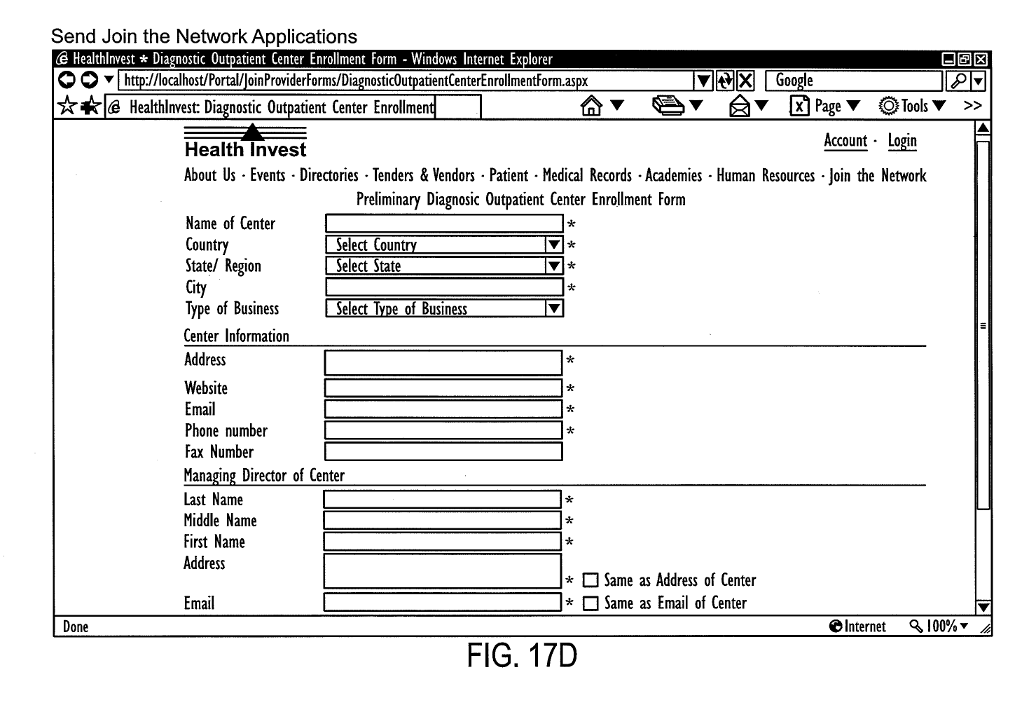

In another aspect of exemplary embodiments of the disclosed system, the global health networking system 100 is configured to allow online authorized/member users to apply for monetary loans with a member financial institution, submit an insurance application to a member insurance company, and submit applications for admission to member medical universities. A user can also submit an application to join a particular network of individuals within the system 100 (e.g., hospital network, physician network, pharmacist network, corporate network, etc.) See, e.g., FIGS. 9A-9D and corresponding screen shot FIGS. 17A-17D.

If a user desires to submit a preliminary loan application to, for example, pay for a medical procedure not covered by an insurance provider (e.g., cosmetic medical procedures), the user chooses the icon designated for loan applications. The system 100 then generates a universal loan application to be completed (see, e.g., FIG. 17A). Once the user completes the preliminary application, the system 100 prompts the user to search for banks by certain search criteria (e.g., country, state, bank name, etc.). The system 100 then searches the plurality of databases 130 for member banks according to the search criteria entered by the user and then displays a list of banks found within the databases 130 that coincide with the search criteria. The user can then select a desired bank. The system 100 is then configured to transmit an email to the selected bank, attaching the loan application, and displays a confirmation and “thank you” message for the user to indicate the email transmission.

Similar to above, with respect to the loan application process, if the user desires to submit a preliminary insurance application for desired insurance coverage, the user can choose the icon designated for insurance applications. The system 100 then prompts the user to choose between four types of insurance coverage and applications, including life insurance, travel insurance, medical insurance, and medical malpractice insurance. Upon the user's selection, the system 100 then generates the universal application to be completed corresponding to the type of coverage the user desires (see, e.g., FIG. 17B). Once the user completes the preliminary application, the system 100 prompts the user to search for insurance companies by certain search criteria (e.g., country, state, insurance company name, etc.). The system 100 then searches the plurality of databases 130 for member insurance companies/providers according to the search criteria entered by the user and then displays a list of the companies/providers found within the databases 130 that coincide with the search criteria. The user can then select a desired company/provider. The system 100 is then configured to transmit an email to the selected insurance company/provider, attaching the application, and displays a confirmation and “thank you” message for the user to indicate the email transmission.

Similar to above, with respect to the loan and insurance application processes, if the user desires to submit a preliminary application for admission to a medical university, the user can choose the icon designated for medical universities. The system 100 then generates a universal application to be completed (see, e.g., FIG. 17C). Once the user completes the preliminary application, the system 100 prompts the user to search for medical universities by certain search criteria (e.g., country, state, name of medical university, etc.). The system 100 then searches the plurality of databases 130 for member medical universities according to the search criteria entered by the user and then displays a list of the universities found within the database that coincide with the search criteria. The user can then select a desired university. The system 100 is then configured to transmit an email to the selected medical university, attaching the application, transcripts, essays, etc., and displays a confirmation and “thank you” message for the user to indicate the email transmission.

If a user desires to join a particular network on the system, the user must submit a preliminary application to join the desired network (see FIG. 9D). The user can choose the icon labeled “Join the Network”. The system 100 then prompts the user to choose between a plurality of networks available (e.g., hospital, physician, pharmacist, corporate, etc.). Upon the user's selection, the system 100 then generates the universal application to be completed corresponding to the network user would like to join (see, e.g., FIG. 17D). The system 100 is then configured to transmit an email to the system administrator, attaching the application, and displays a confirmation and “thank you” message for the user to indicate that the application has been submitted.

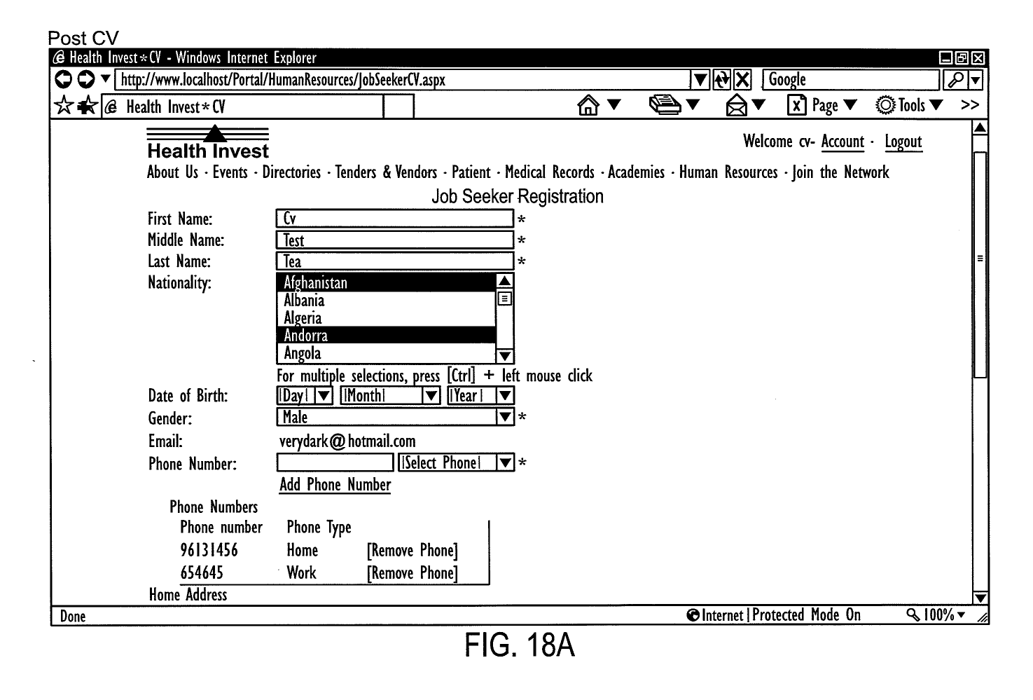

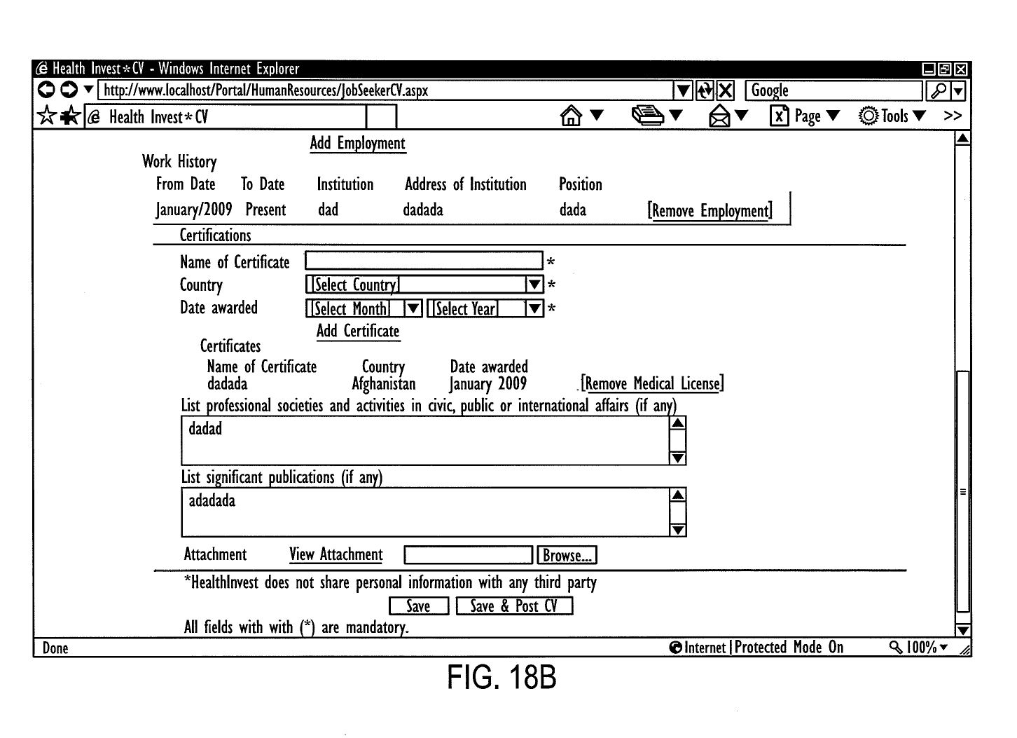

The global health networking system 100 is further configured to receive a curriculum vitae (CV) or a resume from a user (see, e.g., FIG. 10A and corresponding screen shot FIG. 18A). If a user is seeking employment, the system is configured to allow the user to post his/her credentials for viewing. Once the user is logged onto the system 100, the user can choose the icon designated for “Job Seekers”. The system 100 then generates a universal application to be completed (see, e.g., FIGS. 18A and 18B). Once the user completes the preliminary application, the system 100 prompts the user to save the application with any additional desired attachments (e.g., CV, resume, writing samples, transcripts, etc). Once saved, the system 100 prompts the user to search for recruiters by certain search criteria (e.g., country, state, recruiting specialty, etc.). The system 100 then searches the plurality of databases 130 for member recruiters according to the search criteria entered by the user and then displays a list of the recruiters found within the databases 130 that coincide with the search criteria. The user can then review each recruiter's information including his/her credentials, can view the recruiter's website for additional information, and add desired recruiters to a selection. Once recruiters have been selected, the user can post his/her CV/resume recruiters by prompting the system 100 to transmit an email to the selected recruiters, attaching the application, transcripts, essays, etc. The system 100 then displays a confirmation and “thank you” message for the user to indicate that the application has been submitted.

If the user is not member of the global health networking system 100, does not have login information, or account information cannot be verified, the system 100 is configured to prompt the user to register for a new account so the user will be permitted to post his/her credentials.

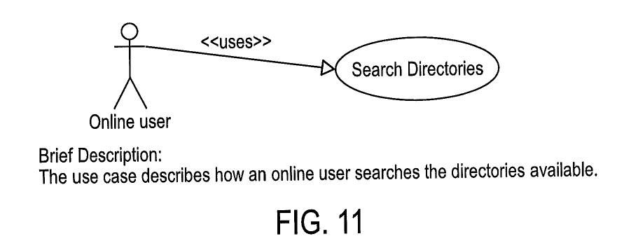

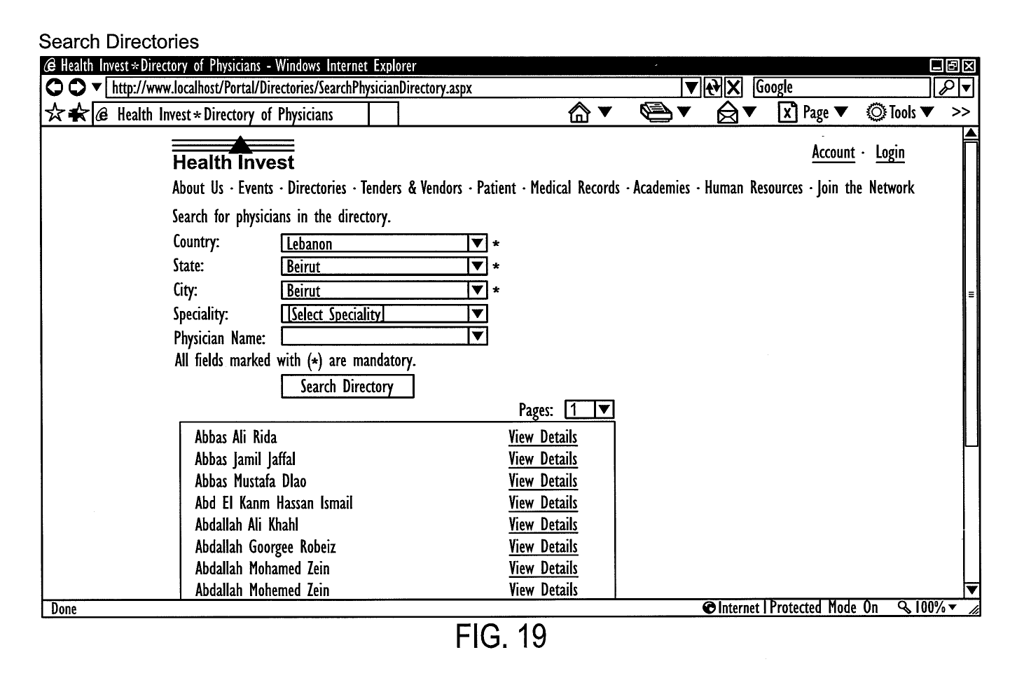

In addition to the above described options, the online user can also search the available directories (see, e.g., FIG. 11 and corresponding screen shot FIG. 19). Once the user is logged onto the system 100, the user can choose the icon designated for “Directories”. The system 100 then prompts the user to choose a directory from an available list of directories (e.g., events, ministries of health, etc.). The system 100 is configured to allow the user to search the desired directory according to various search criteria. The system 100 then displays a list according to the search criteria entered by the user. The user can peruse the details of the list generated by the system 100.

While various exemplary embodiments of the disclosed system have been described above, it should be understood that they have been presented by way of example only, and not limitation. Where methods described above indicate certain events occurring in certain order, the ordering of certain events may be modified. Thus, the breadth and scope of exemplary embodiments of the disclosed system should not be limited by any of the above-described embodiments but should be defined only in accordance with the following claims and their equivalents.

The previous description of the various embodiments is provided to enable any person skilled in the art to make or use the invention recited in the accompanying claims of the disclosed system. While exemplary embodiments of the disclosed system have been particularly shown and described with reference to embodiments thereof, it will be understood by those skilled in the art that many variations, modifications and alternative configurations may be made to the invention without departing from the spirit and scope of exemplary embodiments of the disclosed system.

What is claimed is:

1. A global health networking system comprising:
a global communications network;
at least one server coupled to the global communications network and including a plurality of databases, the plurality of databases including information from multiple countries relating to academic studies, healthcare procedures and drugs, institutes of higher medical related studies, healthcare providers, corporate subscribers, and individuals; the at least one server configured to communicate between each database from the plurality of databases such that users in the global communications network are provided access to information stored in each database for addressing health related topics, said users including patients, healthcare providers, medical students, medical academics and medical device companies and drug companies;
a plurality of user interfaces, each interface from the plurality of interfaces including user selectable options and configured to provide access to the plurality of databases on the at least one server; and
a plurality of search applications configured to search the plurality of databases according to search parameters entered by a user, wherein the user's needs are linked to information stored in the plurality of databases allowing continuous real time access to medical personnel, medical files, and healthcare related information.

2. The system of claim 1, wherein the healthcare providers include health centers, specialty clinics, private and state hospitals, independent physicians, pharmacies, and laboratories.

3. The system of claim 1, wherein the corporate subscribers include academic medical institutions, health product companies, and financial institutions.

4. The system of claim 1, wherein the plurality of databases comprises:
a plurality of datasets configured to provide information on a plurality of data sets including electronic medical records, appointments, retail/products, employee recruitment, medical training, healthcare publications, academic medical centers, public hospitals, private hospitals, specialized medical clinics, physicians, pharmacies, health product companies, medical technicians, lodging, transportation job-seekers, and individuals.

5. The system of claim 1, wherein the plurality of applications include at least a medical search application and an accommodations search application.

6. The system of claim 1, wherein the plurality of applications includes at least a medical search application configured to access information regarding medical services from the healthcare provider database, the medical search application configured to identify medical facilities offering said medical services.

7. The system of claim 1, wherein the plurality of applications includes an accommodations search application configured to access information from the corporate subscriber database regarding at least lodging accommodations and transportation accommodations.

8. The system of claim 1, wherein the plurality of applications allows the user to create a customized medical tour including a desired medical procedure, a desired medical facility, desired transportation accommodations, and desired lodging.

9. The system of claim 1, wherein the plurality of applications includes an individual search application configured to link the user to a plurality of patients from the plurality of databases, such that the user can join a medical tour group with patients seeking similar accommodations according to medical needs.

10. The system of claim 1 wherein the user is medical facility, and wherein the plurality of applications allows the user to create a profile including available elective medical procedures.

11. The system of claim 1, further comprising a live chat program configured to allow a plurality of users to discuss similar medical needs or questions.

12. A system for providing medical related services for a global healthcare network comprising:
a plurality of databases including at least a healthcare provider database, a corporate subscriber database, and a patient database each containing information from multiple regions around the world, wherein each database from the plurality of databases includes a plurality of data sets; and
a plurality of applications, wherein at least one application from the plurality of applications is configured to link the needs of a user with two or more data sets, the data sets including at least electronic medical records, appointments, retail/products, employee recruitment, medical training, healthcare publications, academic medical centers, public hospitals, private hospitals, specialized medical clinics, physicians, pharmacies, health product companies, medical technicians, lodging, transportation, job-seekers, and individuals.

13. A medical tour group system for reserving a medical tour, comprising:
a plurality of databases including at least a healthcare provider database, a corporate subscriber database, and a patient database;
a medical search application configured to access information regarding medical services from the healthcare provider database according to search parameters provided by a user; the medical search application configured to identify medical facilities offering said medical services; and
a plurality of accommodations search applications configured to access information from the corporate subscriber database regarding lodging accommodations and transportation accommodations, wherein a user can create a customized medical tour according to information provided by the user such that the medical tour includes a desired medical procedure, a desired medical facility, desired transportation accommodations, and desired lodging.

14. The medical tour group system of claim 13, wherein the user can be linked to a plurality of patients from the patient database, such that the user can join a medical tour group with patients seeking similar accommodations according to medical needs,

15. The medical tour group system of claim 13, wherein the user can reserve a medical service and a plurality of lodging and transportation accommodations for a plurality of people joining the user on the medical group tour.

16. The medical tour group system of claim 13, further comprising a live chat program configured to allow the user to chat in real time with other users having similar medical needs or questions.

17. A method for reserving a medical tour comprising the steps of:
inputting a user profile into a patient database of the medical tour group system including specific medical and accommodations preferences;
submitting a medical service request to the medical tour group system, wherein a medical search application of the system accesses information regarding medical services from a plurality of databases according to the medical service request and user specific medical preferences; and
submitting an accommodations request to the medical tour group system, wherein a plurality of accommodation search applications access information from the plurality of databases according to the accommodations request and user specific accommodations preferences.

18. The method of claim 17, wherein the medial search request includes at least one of a type of medical procedure, type of medical facility, personal budget, location of facility, and physician/surgeon credential.

19. The method of claim 17, wherein the accommodations search request includes at least one of a destination, transportation accommodations, lodging accommodations, special accommodations, and arrival and departure dates, and number of members in group.

20. The method of claim 17, further comprising the steps of:
submitting a patient request to the medical tour group system, wherein a patient search application accesses authorized patient information from a plurality of databases; and
linking the user to a plurality of patients from the patient database, such that the user can join a medical tour group with patients seeking similar accommodations according to medical needs.

21. The method of claim 17, further comprising the steps of:
receiving medical service information from the medical tour group system according to the medical preferences provided by the user; and
receiving available accommodations information from the medical tour group system according to the accommodation preferences provided by the user.

22. The method of claim 21, wherein the medical service information includes types of medical facilities offering a user desired medical procedure, cost of procedure, location of facility, or physician/surgeon credential.

22. The method of claim 21, wherein the accommodation information includes available transportation accommodations including cost, available lodging accommodations including cost, special accommodations offered.

23. The method of claim 21, further comprising the steps of:
submitting a reservation request to the medical tour group system for selected medical and lodging accommodations.
